SEQUENCE LISTING (12 sequences)

SEQ ID NO: 1 - protein, 9 aa, Artificial sequence
  Synthetic peptide amphiphile
VVVAAAEEE

SEQ ID NO: 2 - protein, 10 aa, Artificial sequence
  Synthetic peptide amphiphile
VVVAAAEEEG

SEQ ID NO: 3 - protein, 10 aa, Artificial sequence
  Synthetic peptide amphiphile
  BINDING (1)..(1): C16 acyl chain
  BINDING (10)..(10): alkyne-modified peptoid
VVVAAAEEEG

SEQ ID NO: 4 - protein, 10 aa, Artificial sequence
  Synthetic peptide amphiphile
  BINDING (1)..(1): C16 acyl chain
  BINDING (9)..(9): (peptoid linker)-(Gd(HP-DO3A) macrocycle
VVVAAAEEEG

SEQ ID NO: 5 - protein, 9 aa, Artificial sequence
  Synthetic peptide amphiphile
  BINDING (1)..(1): C16 acyl chain
  BINDING (9)..(9): NH2
VVVAAAEEE

SEQ ID NO: 6 - protein, 4 aa, Artificial sequence
  Synthetic peptide amphiphile
AAVV

SEQ ID NO: 7 - protein, 6 aa, Artificial sequence
  Synthetic peptide amphiphile
AAAVVV

SEQ ID NO: 8 - protein, 4 aa, Artificial sequence
  Synthetic peptide amphiphile
VVAA

SEQ ID NO: 9 - protein, 6 aa, Artificial sequence
  Synthetic peptide amphiphile
VVVAAA

SEQ ID NO: 10 - protein, 10 aa, Artificial sequence
  Synthetic peptide amphiphile
  BINDING (1)..(1): C16 acyl chain
  BINDING (10)..(10): (peptoid linker)-(Gd(HP-DO3A) macrocycle
VVVAAAEEEG

SEQ ID NO: 11 - protein, 10 aa, Artificial sequence
  Synthetic peptide amphiphile
  BINDING (1)..(1): C16 acyl chain
  BINDING (8)..(8): Gd
VVVVAAAEEE

SEQ ID NO: 12 - protein, 10 aa, Artificial sequence
  Synthetic peptide amphiphile
  BINDING (1)..(1): C16 acyl chain
  BINDING (10)..(10): (GD)3
VVVAAAEEEG

(19) United States
(12) Patent Application Publication
(10) Pub. No.: US 2018/0221513 A1
(43) Pub. Date: Aug. 9, 2018

(54) CONTRAST-AGENT-LABELED PEPTIDE AMPHIPHILE NANOFIBERS

(71) Applicant: Northwestern University, Evanston, IL (US)

(72) Inventors: Adam T. Preslar, Chicago, IL (US); Mark Trosper McClendon, Chicago, IL (US); Samuel I. Stupp, Chicago, IL (US); Thomas J. Meade, Wilmette, IL (US)

(21) Appl. No.: 15/557,994

(22) PCT Filed: Mar. 16, 2016
(86) PCT No.: PCT/US16/22635

§ 371 Date: Sep. 13, 2017

Related U.S. Application Data
(60) Provisional application No. 62/133,725, filed on Mar. 16, 2015.

(51) Int. Cl. A61K 49/14; A61K 51/08

(52) U.S. Cl. CPC A61K 49/14; A61K 51/088

(57) ABSTRACT

Provided herein are compositions and systems comprising contrast-agent (e.g., Gd(III))-labeled peptide amphiphile nanofibers and methods of reporting on biomaterial localization in vivo therewith.

### CROSS REFERENCE TO RELATED APPLICATIONS

The present invention claims the priority benefit of U.S. Provisional Patent Application 62/133,725, filed Mar. 16, 2015, which is incorporated by reference in its entirety.

### STATEMENT REGARDING FEDERAL FUNDING

This invention was made with government support under grant numbers EB005866, P01 HL108795, and U54 CA151880 awarded by the National Institutes of Health. The government has certain rights in the invention.

### FIELD

Provided herein are compositions and systems comprising contrast-agent (e.g., Gd(III))-labeled peptide amphiphile nanofibers and methods of reporting on biomaterial localization in vivo therewith.

### BACKGROUND

Bioactive supramolecular nanostructures are of great importance in regenerative medicine and the development of novel targeted therapies. In order to use supramolecular chemistry to deign such nanostructures, it is extremely important to track their fate in vivo through the use of molecular imaging strategies. Peptide amphiphiles (PAs) are known to generate a wide array of supramolecular nanostructures and their use in areas such as tissue regeneration and therapies for disease are described in the literature.

### SUMMARY

In some embodiments, provided herein are peptide amphiphiles comprising: (a) a hydrophobic non-peptidic segment; (b) a structural peptide segment; (c) a charged peptide segment; and (d) a linker segment.

In some embodiments, the hydrophobic non-peptidic segment is covalently attached to the structural peptide segment, the structural peptide segment is covalently attached to the charged peptide segment; and the charged peptide segment is covalently attached to the linker segment. In some embodiments, the hydrophobic non-peptidic segment is covalently attached to the N-terminus of the structural peptide segment, the C-terminus of the structural peptide segment is covalently attached to the N-terminus of the charged peptide segment; and the C-terminus of the charged peptide segment is covalently attached to the linker segment. In some embodiments, the hydrophobic non-peptidic segment is covalently attached to the C-terminus of the structural peptide segment, the N-terminus of the structural peptide segment is covalently attached to the C-terminus of the charged peptide segment; and the N-terminus of the charged peptide segment is covalently attached to the linker segment.

In some embodiments, the hydrophobic non-peptidic segment comprises an acyl chain of 1 to 25 (e.g., 1, 2, 3, 4, 5, 6, 7, 8, 9, 10, 11, 12, 13, 14, 15, 16, 17, 18, 19, 20, 21, 22, 23, 24, 25, or any ranges there between) carbons in length. In some embodiments, the hydrophobic non-peptidic segment comprises a C₁₆ acyl chain.

In some embodiments, the structural peptide segment is a hydrophobic peptide segment. In some embodiments, the hydrophobic peptide segment comprises histidine (H), isoleucine (I), leucine (L), phenylalanine (F), and/or alanine (A) amino acids. In some embodiments, the structural peptide segment forms hydrogen bonds and/or other stabilizing interactions with a structural peptide segment from an adjacent peptide amphiphile. In some embodiments, the hydrogen bonds and/or other stabilizing interactions result in structure formation that is detectable by circular dichroism and/or microscopy. In some embodiments, the structural peptide segment is a α-helix-forming peptide segment. In some embodiments, the structural peptide segment is a β-sheet-forming peptide segment. In some embodiments, the β-sheet-forming peptide segment comprises 3-8 (e.g., 3, 4, 5, 6, 7, 8, or any ranges there between) histidine (H), isoleucine (I), leucine (L), phenylalanine (F), and/or alanine (A) amino acids. In some embodiments, the β-sheet-forming peptide segment comprises 3-8 valine (V) and alanine (A) amino acids. In some embodiments, the β-sheet-forming peptide segment comprises VVAA (SEQ ID NO: 8). In some embodiments, the β-sheet-forming peptide segment comprises VVVAAA (SEQ ID NO: 9).

In some embodiments, the charged peptide segment comprises acidic and/or basic amino acid residues. In some embodiments, the charged peptide segment comprises 1-6 (e.g., 1, 2, 3, 4, 5 6, or any ranges there between) acidic residues selected from glutamate (E) and aspartate (D). In some embodiments, the charged peptide segment comprises EE, DD, DE, or DD. In some embodiments, the charged peptide segment comprises (X^{a})₃, wherein each X^{a} is an acidic residue. In some embodiments, the charged peptide segment comprises EEE. In some embodiments, the charged peptide segment comprises 1-6 basic residues selected from histidine (H), arginine (R), and lysine (K). In some embodiments, the charged peptide segment comprises (X^{b})₃, wherein each X^{b} is a basic amino acid residue.

In some embodiments, the structural peptide segment and the charged peptide segment comprise VVVAAAEEE (SEQ ID NO: 1). In some embodiments, the structural peptide segment and the charged peptide segment comprise VVVAAAEEEG (SEQ ID NO: 2).

In some embodiments, the linker segment comprises a moiety capable of forming a covalent bond or stable non-covalent bond with a linking agent. In some embodiments, the linker segment comprises a moiety capable of forming a peptide bond with the charged peptide segment. In some embodiments, the linker segment is within the charged peptide segment. In some embodiments, the linker segment comprises a unnatural amino acid, a reactive natural amino acid, a linker peptoid, an antibody-recognizable epitope, or a ligand. In some embodiments, the linker segment comprises a linker peptoid. In some embodiments, the linker peptoid is capable of forming a peptide bond with a standard amino acid residue and displays a linkable moiety (e.g., clickable moiety). In some embodiments, the linkable moiety contains one or more functional groups capable of undergoing a huisgen cycloaddition or alkene hydrothiolation. In some embodiments, the functional group capable of undergoing a huisgen cycloaddition is an alkyne or azide (or other clickable group).

In some embodiments, a peptide amphiphile comprises C₁₆-VVVAAAEEEG-(alkyne-modified peptoid) (SEQ ID NO: 3).

In some embodiments, provided herein are peptide amphiphiles comprising: (a) a hydrophobic non-peptidic segment; (b) a β-sheet-forming peptide segment; (c) an acidic peptide segment; and (d) a linker peptoid. In some embodiments, the hydrophobic non-peptidic segment is covalently attached to the β-sheet-forming peptide segment, the β-sheet-forming peptide segment is covalently attached to the acidic peptide segment; and the acidic peptide segment is covalently attached to the linker peptoid. In some embodiments, the hydrophobic non-peptidic segment is covalently attached to the N-terminus of the β-sheet-forming peptide segment, the C-terminus of the β-sheet-forming peptide segment is covalently attached to the N-terminus of the acidic peptide segment; and the C-terminus of the acidic peptide segment is covalently attached to the linker peptoid.

In some embodiments, the linker peptoid is capable of forming a peptide bond with a standard amino acid residue and displays a linkable moiety. In some embodiments, the linkable moiety is a clickable moiety. In some embodiments, the clickable moiety is an alkyne group. In some embodiments, the alkyne group is displayed on the peptide amphiphile in a manner conducive with reaction of the alkyne with an azide group of a clicking agent to form a covalent bond via click chemistry.

In some embodiments, provided herein are peptide amphiphiles comprising: (a) a hydrophobic non-peptidic segment; (b) a charged peptide segment; and (c) a linker segment. Such peptide amphiphiles lack a structural peptide segment (e.g., C₁₆KKK-linker segment) (See e.g., Leung et al. ACS Nano, 2012, 6 (12), pp 10901-10909; incorporated by reference in its entirety). Any suitable features or elements described herein for use with peptide amphiphiles having a structural peptide segment may be applied to peptide amphiphiles lacking such a segment.

In some embodiments, provided herein are compositions comprising a peptide amphiphile described herein (e.g., described above and/or in the Detailed Description) and a contrast agent comprising: (a) (i) a linking moiety covalently attached to a (i) chelation moiety; and (b) a metal ion (e.g., Gd(III)). In some embodiments, the linking moiety of the contrast agent is capable of forming a covalent bond or stable non-covalent bond with the linker segment of the peptide amphiphile. In some embodiments, the linking moiety of the contrast agent comprises one or more functional groups capable of undergoing a huisgen cycloaddition or alkene hydrothiolation with the linker segment of the peptide amphiphile (e.g., clickable and clicking groups). In some embodiments, the linking moiety of the contrast agent and the linker segment of the peptide amphiphile are: (i) an alkyne and azide, or (ii) an azide and alkyne, respectively. In some embodiments, the linking moiety comprises an azide group. In some embodiments, chelation moiety is selected from the group consisting of EDTA, DTPA, TTHA, DOTA, TAGA, DOTP, DTPA-BMA, DO2P, and HP-DO3A. In some embodiments, the chelation moiety comprises HP-DO3A. In some embodiments, the metal ion is selected from the group consisting of Mn(II), Gd(III), Dy(III), Ho(III), Er(III), Eu(III), Tb(III), Sm(III), Ce(III), Pr(III), Yb(III), Tm(III), Nd(III), and Tb(IV). In some embodiments, the metal ion comprises Gd(III). In some embodiments, the contrast agent comprises and azide-linked Gd(HP-DO3A) macrocycle.

In some embodiments, provided herein are contrast-agent-labeled peptide amphiphiles comprising the reaction product of a contrast agent (e.g., described above and/or in the Detailed Description) and peptide amphiphile (e.g., described above and/or in the Detailed Description). In some embodiments, the contrast-agent-labeled peptide amphiphile of comprises a C₁₆-VVVAAAEEE-(peptoid linker)-(Gd(HP-DO3A) macrocycle) (SEQ ID NO: 4). In some embodiments, the contrast-agent-labeled peptide amphiphile of comprises a C₁₆-VVVAAAEEEG-(peptoid linker)-(Gd(HP-DO3A) macrocycle) (SEQ ID NO: 10). In some embodiments, the peptoid linker and (Gd(HP-DO3A) macrocycle are covalently attached via alkyne/azide chemistry. In some embodiments, the contrast-agent-labeled peptide amphiphile comprises PA1, PA2, PA3, PA4, or variants thereof.

In some embodiments, provided herein are nanofibers comprising contrast-agent-labeled peptide amphiphiles (e.g., described above and/or in the Detailed Description) and further comprising peptide amphiphiles not-labeled with a contrast agent. In some embodiments, the contrast-agent-labeled peptide amphiphiles are Gd(III)-labeled peptide amphiphiles and the peptide amphiphiles not-labeled with a contrast agent are un-Gd(III)-labeled peptide amphiphiles. In some embodiments, at least 50% (e.g., 50%, 55%, 60%, 65%, 70%, 75%, 80%, 85%, 90%, 95%, 99%, or any ranges there between) of the nanofiber is the peptide amphiphiles not-labeled with a contrast agent.

In some embodiments, provided herein are nanofibers comprising contrast-agent-labeled peptide amphiphiles (e.g., described above and/or in the Detailed Description) and further comprising bioactive peptide amphiphiles (e.g., displaying a bioactive moiety (e.g., a bioactive epitope), not-labeled with a contrast agent). Exemplary peptide amphiphiles displaying bioactive groups (e.g., epitopes) are described in, for example, U.S. Pat. No. 8,834,840; U.S. Pat. No. 8,772,228; U.S. Pat. No. 8,748,569; U.S. Pat. No. 8,580,923; U.S. Pat. No. 8,512,693; U.S. Pat. No. 8,450,271; U.S. Pat. No. 8,138,140; U.S. Pat. No. 8,124,583; U.S. Pat. No. 8,114,835; U.S. Pat. No. 8,114,834; U.S. Pat. No. 8,080,262; U.S. Pat. No. 8,063,014; U.S. Pat. No. 7,851,445; U.S. Pat. No. 7,838,491; U.S. Pat. No. 7,745,708; U.S. Pat. No. 7,683,025; U.S. Pat. No. 7,554,021; U.S. Pat. No. 7,544,661; U.S. Pat. No. 7,534,761; U.S. Pat. No. 7,491,690; U.S. Pat. No. 7,452,679; U.S. Pat. No. 7,390,526; U.S. Pat. No. 7,371,719; incorporated by reference in their entireties.

In some embodiments, provided herein are nanofibers comprising: (i) contrast-agent-labeled peptide amphiphiles (e.g., described above and/or in the Detailed Description), (ii) bioactive peptide amphiphiles (e.g., displaying a bioactive moiety (e.g., a bioactive epitope) and not-labeled with a contrast agent), and (iii) filler peptide amphiphiles (e.g., not-labeled with a contrast agent and not displaying a bioactive moiety. In some embodiments, nanofibers comprise: (i) less than 50% (e.g., 49%, 45%, 40%, 35%, 30%, 25%, 20%, 15%, 10%, 5%, 4%, 3%, 2%, 1%, or any ranges there between) contrast-agent-labeled peptide amphiphiles; (ii) less than 50% (e.g., 49%, 45%, 40%, 35%, 30%, 25%, 20%, 15%, 10%, 5%, 4%, 3%, 2%, 1%, or any ranges there between) bioactive peptide amphiphiles; and at least 2% (e.g., 2%, 3%, 4%, 5%, 10%, 15%, 20%, 25%, 30%, 35%, 40%, 45%, 50%, 55%, 60%, 65%, 70%, 75%, 80%, 85%, 90%, 95%, 98%, or any ranges there between) filler peptide amphiphiles. In some embodiments, nanofibers comprise at least 50% (e.g., 50%, 55%, 60%, 65%, 70%, 75%, 80%, 85%, 90%, 95%, 98%, or any ranges there between) filler peptide amphiphiles.

In some embodiments, provided herein are methods of monitoring biomaterials in vivo comprising administering a composition comprising the nanofibers, compositions, and/or peptide amphiphiles described herein to a human or animal subject as an in vivo implant label and monitoring by a biophysical technique. In some embodiments, the biophysical technique is magnetic resonance imaging (MRI). In some embodiments, the biophysical technique is mass spectroscopy. In some embodiments, the biophysical technique is a radioimaging technique. In some embodiments, the radioimaging technique is Positron emission tomography (PET) or single-photon emission computed tomography (SPECT).

Provided herein are compositions comprising nanofibers of Gd(III)-labeled peptide amphiphiles. In some embodiments, the nanofibers further comprise un-Gd(III)-labeled peptide amphiphiles. In some embodiments, greater than 50% of the nanofiber is un-Gd(III)-labeled peptide amphiphiles (e.g., >55%, >60%, >65%, >70%, >75%, >80%, >85%, >90%, >95%, >96%, >97%, >98%, >99%). In some embodiments, less than 100% of the nanofiber is un-Gd(III)-labeled peptide amphiphiles (e.g., <99%, <98%, <97%, <96%, <95%, <90%, <85%, <80%, <75%, <70%, <65%, <60%). In some embodiments, greater than 1% of the nanofiber is Gd(III)-labeled peptide amphiphiles (e.g., >1%, >2%, >3%, >4%, >5%, >10%, >15%, >20%, >25%, >30%, >35%, >40%, >45%). In some embodiments, less than 50% of the nanofiber is Gd(III)-labeled peptide amphiphiles (e.g., <45%, <40%, <35%, <30%, <25%, <20%, <15%, <10%, <5%, <4%, <3%, <2%). In some embodiments, any suitable ranges of Gd(III)-labeled peptide amphiphiles and un-Gd(III)-labeled peptide amphiphiles (e.g., as defined by the above limits) find use in embodiments herein. In some embodiments, the peptide amphiphiles comprise a peptide sequence comprising one or more V, E, and/or A residues (e.g., VₓA_{y}E_{z}, wherein x, y, and z are 0-5, and wherein rearrangements of the order of V, A, and/or E are allowed. In some embodiments, the peptide amphiphiles comprise a peptide sequence comprising X₃₋₁₂, wherein each X is independently selected from: V, A, and E. In some embodiments, the Gd(III)-labeled peptide amphiphiles comprises C₁₆V₃A₃E₃-NH₂. In some embodiments, the un-Gd(III)-labeled peptide amphiphiles comprises C₁₆V₃A₃E₃-NH₂. In some embodiments, peptide amphiphiles comprise PA1, PA2, PA3, PA4, or variants thereof.

### BRIEF DESCRIPTION OF THE DRAWINGS

FIGS. 1A-B. Summary of chelate conjugation chemistry and the compounds investigated in this work A) Click addition of azide-modified Gd(HPN₃DO₃A) to the alkyne peptoid was performed in solution a B) Chemical structures of MR contrast agents based on conjugation of the V3A3 peptide sequence.

FIGS. 2A-C. A) Small angle X-ray scattering (SAXS) data for each PA when dissolved in 10 mM Tris buffer (bottom), after thermal annealing (middle), and after the addition of CaCl₂ to thermally annealed solutions (top). B) Cryo-TEM of the same conjugates after thermal annealing (scale bar is 200 nm). C) Molecular graphics representation of the various peptide amphiphile assemblies.

FIGS. 3A-B. Nuclear Magnetic Resonance Dispersion (NMRD) profiles for all PAs at three different conditions. All profiles were collected at 37° C. with a PA concentration of 2 mM. The fits for PA4 in annealed and buffered conditions are nearly identical.

FIG. 4A-B. Relaxation time measurements of PA solutions for PA1 and PA3 in Tris buffer at 7 T. A) Summary of T₁ values of mixtures of PA1 and PA3 with the filler sequence C₁₆V₃A₃E₃-NH₂ (SEQ ID NO: 5). B) Summary of T₂ values of mixtures of PA1 and PA3 with the filler sequence C₁₆V₃A₃E₃-NH₂(SEQ ID NO: 5). The short T₂ for PA3 confirms that T₂ relaxation is dominating T₁ relaxation of PA3.

FIG. 5. Summary of in vivo measurements of PA1 and PA3 in a murine leg model. A) Anatomical scan of mouse legs immediately after injection (top row) and after four days (bottom row). The PA injections are indicated by white arrows. PA1 produces positive contrast in white (left column) while PA3 produces negative contrast (right column). B) T₁ maps of the same mouse at the same image positions as in A. Dark areas represent regions with very short T₁ times. C) Averaged image T₁ times from regions of interest for all mice at all slices where PA was visible. Filler (unlabeled) PA was not visible. Filler PA T₁ was measured as a best approximation for PA position based on the injection location. Background was measured by averaging T₁ values of muscle tissue several mm from the PA injection.

FIG. 6. Cryo-EM images of each contrast agent PA after dissolution in 8 mM Tris buffer at pH 7.4. Structures indicated by black arrows. A) PA1 B) PA2 C) PA3 D) PA4.

FIG. 7. Circular Dichroism of PA compounds. Bisignate peaks around 200 nm and 216 are indicative of a betasheet morphology. All measurements performed at 0.1 mMagent in 10 mM Tris buffered 50 mM NaCl at pH 7.4.

FIGS. 8A-C. Tracking PA nanofibers in vivo. (A) PAs containing a chelated Gd ion (1P or 3P) were injected into non-injured mice hearts and then imaged at days 2-3 using Magnetic Resonance Imaging (MRI). Arrowheads indicate the presence of PA nanofibers. (B) A fluorescently-labeled PA (with TRITC) was injected in non-injured hearts and its presence was confirmed at days 0 and 3 after injection. (C) Some of the hearts injected with the TRITC-labeled PA were used to image using an IVIS (In vivo Imaging System, Perkin Elmer). The heart on the left and the heart on the middle were injected with the PA, while the one in the right was not.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“Octan”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence “having at least Y % sequence identity with SEQ ID NO:Z” may have up to X substitutions relative to SEQ ID NO:Z, and may therefore also be expressed as “having X or fewer substitutions relative to SEQ ID NO:Z.”

As used herein, the term “nanofiber” refers to an elongated or threadlike filament (e.g., having a significantly greater length dimension that width or diameter) with a diameter typically less than 100 nanometers.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the term “physiological conditions” refers to the range of conditions of temperature, pH and tonicity (or osmolality) normally encountered within tissues in the body of a living human.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a non-peptide lipophilic (hydrophobic) segment, a structural peptide segment and/or charged peptide segment (often both), and optionally a functional segment (e.g., linker segment, bioactive segment, etc.). The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptidic segment (e.g., comprising an acyl group of six or more carbons), (2) a β-sheet-forming peptide segment; (3) a carboxyl-rich peptide segment, and (4) a functional segment (e.g., linker segment).

As used herein and in the appended claims, the term “lipophilic moiety” or “hydrophobic moiety” refers to the moiety (e.g., an acyl moiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and micelle (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)— where n=2-25. In some embodiments, a linear acyl chain is the lipophilic group, palmitic acid. However, other small lipophilic groups may be used in place of the acyl chain.

As used herein, the term “structural peptide” refers to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural segments of adjacent structural segments. In some embodiments, nanofibers of peptide amphiphiles having structural peptide segments display linear or 2D structure when examined by microscopy and/or α-helix and/or β-sheet character when examined by circular dichroism (CD).

As used herein, the term “beta (β)-sheet-forming peptide segment” refers to a structural peptide segment that has a propensity to display (β)-sheet-like character (e.g., when analyzed by CD). In some embodiments, amino acids in a beta (β)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the terms “carboxy-rich peptide segment,” “acidic peptide segment,” and “negatively-charged peptide segment” refer to a peptide sequence of a peptide amphiphile that comprises two or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues with acidic side chains could be used, as will be evident to one ordinarily skilled in the art. There may be from about 2 to about 7 amino acids, and or about 3 or 4 amino acids in this segment.

As used herein, the terms “amino-rich peptide segment,” “basic peptide segment,” and “positively-charged peptide segment” refer to a peptide sequence of a peptide amphiphile that comprises two or more amino acid residues that have side chains displaying positively-charged acid side chains (e.g., Arg (R), Lys (K), His (H), or non-natural amino acids). A basic peptide segment may optionally contain one or more additional (e.g., non-basic) amino acid residues. Non-natural amino acid residues with acidic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate a bioactivity. Peptide amphiphiles and structures (e.g., nanofibers) bearing functional peptides exhibit the functionality of the functional peptide.

As used herein, the term “linkable” refers to a compound or moiety that forms a covalent bond or stable noncovalent bond when contacted or in proximity of a “linking” compound or agent. The “linking” compound or moiety comprises a reactive portion which forms the bond with the linkable compound or moiety and a functional portion which facilitates detection or isolation of a tagged compound or complex. The term “clickable” refers to a compound or moiety displaying a functional group that enables tagging with a “clicking” compound or moiety via click chemistry (e.g., via huisgen cycloaddition or alkene hydrothiolation). Suitable clickable/clicking pairs include, for example, azide/alkyne and thiol/alkene.

### DETAILED DESCRIPTION

Provided herein are compositions and systems comprising contrast-agent (e.g., Gd(III))-labeled peptide amphiphile (PA) nanofibers and methods of reporting on biomaterial localization in vivo therewith. In some embodiments, peptide amphiphiles are functionalized (e.g., via a linker segment) with an imaging agent (e.g., contract agent) for use in whole body imaging (e.g., magnetic resonance imaging (MRI), single photon emission computed tomography (SPECT), etc.). In particular embodiments, provided herein are PA molecules based on, for example, the structural segment (e.g., β-sheet amino acid sequence) V₃A₃ conjugated to macrocyclic Gd(III) labels for magnetic resonance imaging (MRI). These conjugates were shown to form cylindrical supramolecular assemblies using cryogenic transmission electron microscopy (Cryo-EM) and small angle X-ray scattering (SAXS). Using nuclear magnetic relaxation dispersion (NMRD) analysis, it was observed that thermal annealing of the nanostructures led to a decrease in water exchange lifetime (τ_{M}) of hundreds of nanoseconds only for molecules that self-assemble into nanofibers of high aspect ratio. This decrease indicates more solvent exposure of the paramagnetic moiety on annealing, resulting in faster water exchange within angstroms of the macrocycle. Two of the self-assembling conjugates were imaged after intramuscular injections of the PA C₁₆V₃A₃E₃-NH₂ in the tibialis anterior muscle in a murine model. Needle tracts were clearly discernible with MRI at four days post-injection. This work establishes Gd(III) macrocycle-conjugated peptide amphiphiles as effective tracking agents for peptide amphiphile materials in vivo over the timescale of days. However, such exemplary peptide amphiphiles should not be viewed as limiting on the scope herein. Other peptide amphiphile sequences (e.g., other structural segments, other charged segments, etc.), linker chemistries, and contrast agents will be understood based on the disclosure herein.

Supramolecular self-assembly offers biomimetic strategies to produce complex nanostructures for applications across many chemical and biological applications (refs. 1-4; incorporated by reference in their entireties). Self-assembly has been used to direct regeneration of tissues such as cartilage (ref 5; incorporated by reference in its entirety) and bone (ref 6; incorporated by reference in its entirety), and blood vessels (refs. 7-10; incorporated by reference in their entireties). Compared to artificial systems, nanostructures based on biological building blocks have the advantage of biocompatibility as well as natural degradation pathways. In this context, peptide amphiphile nanostructures are useful to design bioactive nanostructures (ref 11; incorporated by reference in its entirety).

Peptide amphiphiles (PAs) that self-assemble into well-defined nanoscale fibrous structures emulating extracellular matrices are promising in many regenerative medicine applications (ref 12; incorporated by reference in its entirety). Their relatively short peptide sequences are covalently conjugated to hydrophobic fatty acid tails. The peptide sequences are typically composed of charged residues for solubility and in some cases a bioactive terminus to be displayed to the biological environment. Sequences of amino acids with high beta-sheet propensities promote assembly into one-dimensional nanostructures in water. In solution, the PAs described self-assemble into nanostructures with varying one-dimensional morphologies and physical properties that depend on their specific design and assembly conditions (refs. 13-16; incorporated by reference in their entireties). The platform is highly versatile because it allows the incorporation of specific functions without disrupting fiber-like morphologies. Furthermore, the density of these functional structures at their termini on fiber surfaces is controllable through co-assembly with non-functional molecules (refs. 9, 17-19; incorporated by reference in their entireties). Fibrous PA structures delivered to biological tissues are designed to perform their function, and then biodegrade into natural building blocks. However, this degradation process, so important for understanding material function and clearance, is not well understood in vivo. MRI strategies to image PA spatiotemporal presence in vivo are a critical tool for their further development as therapies (refs. 20-22; incorporated by reference in their entireties).

Single-photon emission computed tomography (SPECT) is a nuclear medicine tomographic imaging technique using gamma rays. It is similar to conventional nuclear medicine planar imaging using a gamma camera; however, it is able to provide true 3D information, which is typically presented as cross-sectional slices through the patient. The technique relies on the delivery of a gamma-emitting radioisotope (a radionuclide, such as radiogadolinium) into the patient. In some embodiments, a marker radioisotope is attached to a specific ligand to create a radioligand, whose properties bind it to certain types of tissues. This marriage allows the combination of ligand and radiopharmaceutical to be carried and bound to a place of interest in the body, where the ligand concentration is seen by a gamma camera.

Magnetic resonance imaging (MRI) is ideally suited to provide long-term imaging with high spatial resolution without exposing the subject to ionizing radiation. Signal intensity in MR imaging is dependent on proton relaxation rates, field strength and acquisition sequence (ref 23; incorporated by reference in its entirety). MR contrast agents accelerate magnetic relaxation to increase contrast (ref 24,25; incorporated by reference in their entireties). Among T₁ contrast agents, Gd(III) macrocycles have shown great clinical and research success owing to their stability and high magnetic moments (ref 26; incorporated by reference in its entirety). Previously, contrast agent-PA conjugates were developed for magnetic resonance imaging (MRI) and observed that these compounds exhibited enhanced image contrast due to their slow molecular tumbling rate (refs. 27, 28; incorporated by reference in their entireties). A series of contrast agent PAs in which the steric and electrostatic repulsions among the macrocyclic contrast agents drove changes in assembly morphology that depend on pH have been developed (ref 29; incorporated by reference in its entirety).

Nuclear magnetic relaxation dispersion (NMRD) profiles, which measure proton relaxation time as a function of magnetic field strength (ref 30; incorporated by reference in its entirety), provide insight into the interplay of macrocycle rotational dynamics (TR) and water exchange lifetime (τm), among other parameters (refs. 26, 31-33; incorporated by reference in their entireties). The Florence NMRD model, developed to analyze the NMRD profiles when deviations from the Solomon-Bloembergen-Morgan (SBM) theory are expected due to the presence of ZFS, has been used to interpret the profiles by modeling water relaxation by Gd(III) macrocycles (refs. 30, 34-36; incorporated by reference in its entirety). From these profiles, the parameters τR and τm are extracted. With a mechanistic understanding of a contrast agent, agent sensitivity is increased through its molecular design.

Experiments were conducted during development of embodiments of the present invention to develop self-assembling PAs conjugated at different positions with Gd(III) contrast agents as in vivo implant labels and use NMRD to understand how assembly conditions affect the parameters of water relaxation, particularly τM. Four exemplary compounds were designed to test the effects of sterics and radial placement of the gadolinium chelate on relaxivity. A chelate based on a clinically approved agent was chosen for its high stability (ref 37; incorporated by reference in its entirety). This is particularly important when considering clinical applications and long clearance periods. PA compounds were assessed as contrast agents using relaxation measurements after dissolving in buffer, thermally annealing, and after cross-linking annealed solutions with CaCl₂. and then used to fate-map implanted PA gels in the tibialis anterior muscle of the mouse limb over days.

In some embodiments, provided herein are supramolecular MRI contrast agent-peptide amphiphile conjugates, and methods of using the same to investigate the relationship between nanostructure morphology and MR contrast agent relaxivity. PA1-4 all showed high, τ_{R}-modulated, relaxivity with optimum compounds and conditions producing relaxivities greater than 20 mM⁻¹s⁻¹ at 60 MHz. Thermal annealing and the presence of Ca(II) ions in solution increased relaxivity of conjugates that formed long fibers. These changes are a result of water exchange lifetime shortening, providing a new route to controlling water exchange on the surface of contrast agent nanostructures. Gd(III)-PA complexes were used to fate-map a PA gel in an in vivo mouse model, showing persistence over a 4-day period. The supramolecular structures formed by self-assembly of these Gd-PAs provide a useful approach to track the fate of biomaterials over time after they have been implanted in vivo.

The technology described herein improves on the detection of, for example, peptide hydrogels in vivo, providing a proxy for biomaterial breakdown and tissue health, important variables for assessing regenerative treatment success. In some embodiments, the compositions improve hydrogel detection by relaxation mapping (T₁ or T₂) and anatomical imaging. In some embodiments, the compositions are used to enhance MR sensitivity when measuring local fluid diffusion anisotropy, as with diffusion tensor imaging (DTI).

In some embodiments, the technology is specific to peptide amphiphile systems. In some embodiments, sequence homology between the MRI probe (the Gd(III) agent) and the biomaterial promote nanostructure integrity. In some embodiments, macroscopic gel properties are minimally disrupted, even with a large (2-10 mol %) Gd(III) payload. In some embodiments, competing imaging systems rely on nonhomologous peptide sequences and often label the nanostructures chemically after they are formed. The result is non-quantitative and disruptive to nanostructure. Experiments conducted during development of embodiments of the present invention have shown a high per-Gd(III) relaxivity for this system, indicating a greater probe efficiency. Experiments conducted during development of embodiments of the present invention have applied the materials with specific protocols and used them to image the gel in vivo.

In some embodiments, provided herein are compositions, systems, and methods for tracking peptide amphiphiles in vivo. In some embodiments, MRI is used because it allows multiple imaging sessions without risk to the patient and has infinite tissue penetration depth. Regenerative medicine technologies often depend on hydrogel materials, which have very poor visibility in an MR image. To correlate biomaterial breakdown, tissue regeneration, and subject (or patient) outcomes, it must be possible to visualize biomaterial location and breakdown. Additionally, the agents may facilitate more complex measurements, such as diffusion anisotropy, to correlate with tissue health.

Magnetic resonance imaging of self-assembled biomaterial scaffolds are described, for example, in U.S. Pat. No. 8,834,840, which is herein incorporated by reference in its entirety. Preslar, et al. ACS Nano. 2014 Jul. 22; 8(7):7325-32. Is also herein incorporated by reference in its entirety.

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus of the peptide, in order to create the lipophilic segment. Synthesis typically starts from the C-terminus, to which amino acids are sequentially added using either a Rink amide resin (resulting in an —NH₂ group at the C-terminus of the peptide after cleavage from the resin), or a Wang resin (resulting in an —OH group at the C-terminus). Accordingly, embodiments described herein encompasses peptide amphiphiles having a C-terminal moiety that may be selected from the group consisting of —H, —OH, —COOH, —CONH₂, and —NH₂.

The lipophilic or hydrophobic segment is typically incorporated at the N-terminus of the peptide after the last amino acid coupling, and is composed of a fatty acid or other acid that is linked to the N-terminal amino acid through an acyl bond (although embodiments herein are not limited to such methods). In aqueous solutions, PA molecules self-assemble (e.g., into cylindrical micelles (a.k.a nanofibers)) that bury the lipophilic segment in their core and display the functional peptide on the surface. The structural peptide undergoes intermolecular hydrogen bonding to form beta sheets that orient parallel to the long axis of the micelle.

In some embodiments, compositions described herein comprise PA building blocks that in turn comprise a hydrophobic segment and a peptide segment. In certain embodiments, a hydrophobic (e.g., hydrocarbon and/or alkyl tail) segment of sufficient length (e.g., 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, or more, or any ranges there between) is covalently coupled to peptide segment (e.g., a peptide comprising a segment having a preference for beta-strand conformations) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture. In various embodiments, hydrophobic segments pack in the center of the assembly with the peptide segments exposed to an aqueous or hydrophilic environment to form cylindrical nanostructures that resemble filaments. Such nanofilaments display the peptide regions on their exterior and have a hydrophobic core.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, aromatic segments, pi-conjugated segments, etc. In some embodiments, the hydrophobic segment comprises an acyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25)

In some embodiments, peptide amphiphiles comprise a functional moiety or bioactive moiety. In particular embodiments, a functional moiety is the C-terminal most segment of the PA. In some embodiments, the functional moiety is attached to the C-terminal end of a charged segment (e.g., [hydrophobic segment]-[structural segment]-[charged segment]-[functional moiety]). In other embodiments, a functional moiety is the N-terminal most segment of the PA. In other embodiments, a functional moiety is appended onto one of the other segments of a PA. In some embodiments, the functional moiety is exposed on the surface of an assembled PA structure (e.g., nanofiber). In some embodiments, the functional moiety is a peptide, but is not limited thereto. Functional peptides and other moieties for achieving such functionality will be understood.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises two or more (e.g., 2, 3, 4, 5, 6, 7, or more) acidic residues (D and/or E) in sequence. In some embodiments, the acidic peptide segment comprises up to 7 residues in length and comprises at least 50% acidic residues.

In some embodiments, peptide amphiphiles comprise a basic peptide segment. For example, in some embodiments, the acidic peptide comprises two or more (e.g., 2, 3, 4, 5, 6, 7, or more) basic residues (R, H, and/or K) in sequence. In some embodiments, the basic peptide segment comprises up to 7 residues in length and comprises at least 50% basic residues.

In some embodiments, peptide amphiphiles comprises a structural and/or beta-sheet-forming segment. In some embodiments, the structural segment is rich in H, I, L, F, V, and A residues. In some embodiments, the structural and/or beta-sheet-forming segment comprises an alanine- and valine-rich peptide segment (e.g., AAVV (SEQ ID NO: 6), AAAVVV (SEQ ID NO: 7), or other combinations of V and A residues, etc.). In some embodiments, the structural and/or beta sheet peptide comprises 4 or more consecutive A and/or V residues, or conservative or semi-conservative substitutions thereto. In some embodiments, the structural and/or beta-sheet forming peptide segment comprises 4 or more consecutive non-polar aliphatic residues (e.g., alanine (A), valine (V), leucine (L), isoleucine (I), methionine (M)).

In some embodiments, the structural and/or beta-sheet forming peptide segment comprises 4-16 amino acids in length and comprises 4 or more (e.g., 4, 5, 6, 7, 8, 9, 10, 11, 12, 13, 14, 15, 16, or ranges there between) non-polar aliphatic residues.

In some embodiments, PA molecules comprise a linker segments. The linker segment provides a location on the PA for attachment of a contract agent (e.g., comprising a complementary linking moiety). Embodiments herein describe the use of a linker peptoid for attachment of a contrast agent by, for example, click chemistry. Embodiments herein are not limited to peptoids or to such chemistries. In some embodiments, a linker segment is a peptide comprising a reactive natural amino acid (e.g., Lys (K), Arg (R), etc.), unnatural amino acid having a reactive group, epitope sequence (e.g., recognizable by an antibody or other immunogenic agent), a ligand (e.g., coordinated by a binding agent), etc.

In some embodiments, PA molecules herein comprise a linker peptoid (e.g., at the C-terminus). In some embodiments, a linker peptoid is an agent or moiety capable for forming a peptide bond with an amino acid reside and displaying a linkable group (e.g., a clickable group (e.g., an alkyne, etc.), etc.). In some embodiments, the linkable group forms a stable bond (e.g., covalent bond when contacting a corresponding linking agent (e.g., clicking agent (e.g., azide, etc.), etc.). Suitable linkable moiety/linking agent pairs are functional groups or compounds that rapidly and reliably form stable interactions under biological-like conditions. Exemplary pairs are alkyne/azide, thiol/maleimide, thiol/haloacetyl (e.g., iodoacetyl, etc.), thiol/pyridyl disulfide (e.g. pyridyldithiol, etc.), sulphonyl azides/thio acids, etc. In some embodiments, either member of the pair may find use as the linkable moiety (e.g., on the linking peptoid) or the linking agent (e.g., comprising the gadolinium moiety). Other functional-group pairs that form covalent bonds or other stable interactions may also be used.

In some embodiments, a linkable moiety (e.g., of a linking peptoid) is a clickable moiety, capable of rapid covalent bonding with a clicking agent (e.g., reactive portion of a clicking agent). Exemplary pairs of clickable moieties and clicking agents include, but are not limited to alkyne and azide groups, transcyclooctene and tetrazine groups, dibenzocyclooctyne and azide groups, etc. In some embodiments, either member of the pair may find use as the clickable moiety (e.g., of a linking peptoid) or the clickable agent. Other functional-group pairs capable of click chemistry may also be used.

In some embodiments, provided herein are PA molecules linked to a contrast agent. In some embodiments, a contrast moiety is attached to a linking agent (e.g., clicking agent). In some embodiments, reaction of the linking agent with the linkable moiety (e.g., clickable moiety) of the PA results in conjugation of the contrast agent to the PA.

In some embodiments, a contrast agent comprises a chelation moiety coordinated with a metal ion or metal ion cluster capable of providing contrast in an appropriate detection scheme (e.g., magnetic resonance imaging (MRI)). Suitable metal ions or clusters may be selected from:

manganese, a lanthanide, iron-oxide particles, etc. In embodiments in which the metal ion is a lanthanide, the lanthanide may be selected from the group consisting of: Gd(III), Dy(III), Ho(III), Er(III), Eu(III), Tb(III), Sm(III), Ce(III), Pr(III), Yb(III), Tm(III), Nd(III), and Tb(IV). In some embodiments, the contrast agent comprises a chelation moiety, suitable chelation moieties may be selected from the group consisting of EDTA, DTPA, TTHA, DOTA, TAGA, DOTP, DTPA-BMA, DO2P, and HP-DO3A. Any suitable combination of metal ion or metal ion cluster and chelation moiety may find use in embodiments herein. In some embodiments, the contrast agent comprises: (i) a linking moiety (e.g., azide) covalently attached to a chelation moiety (e.g., HP-DO3A), and (ii) a metal ion (e.g., Gd(III)).

Suitable peptide amphiphiles, PA segments, PA nanostrcutures, and associated reagents and methods are described, for example in U.S. patent application Ser. No. 8,512,693; U.S. patent application Ser. No. 8,450,271; U.S. patent application Ser. No. 8,138,140; U.S. patent application Ser. No. 8,124,583; U.S. patent application Ser. No. 8,114,835; U.S. patent application Ser. No. 8,114,834; U.S. patent application Ser. No. 8,080,262; U.S. patent application Ser. No. 8,063,014; U.S. patent application Ser. No. 7,851,445; U.S. patent application Ser. No. 7,838,491; U.S. patent application Ser. No. 7,745,708; U.S. patent application Ser. No. 7,683,025; U.S. patent application Ser. No. 7,554,021; U.S. patent application Ser. No. 7,544,661; U.S. patent application Ser. No. 7,534,761; U.S. patent application Ser. No. 7,491,690; U.S. patent application Ser. No. 7,452,679; U.S. patent application Ser. No. 7,390,526; U.S. patent application Ser. No. 7,371,719; U.S. patent application Ser. No. 6,890,654; herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural segment, functional segment, etc.). For example, nanofibers, nanospheres, intermediate shapes, and other supramolecular structures are achieved by adjusting the identity of the PA component parts.

### EXPERIMENTAL

### Example 1

¹NMRD profiles were collected with a Stelar Spinmaster FFC-2000-1T fast field cycling relaxometer in the 0.01-40 MHz proton Larmor frequency range, at 293 and 310 K. Longitudinal relaxation rates were measured with an error smaller than 1%. Proton relaxivities were calculated by subtracting the diamagnetic contribution of the peptide nanofibers in the absence of Gd(III) from the relaxation rates of the paramagnetic samples, and normalizing to 1 mM Gd(III) concentration. The profiles were analyzed using the Freed model for outer-sphere relaxation (refs. 48, 49; incorporated by reference in their entireties) and the modified Florence NMRD program for the inner-sphere relaxation (refs. 45, 46, 50; incorporated by reference in their entireties). The effect of static ZFS on nuclear relaxation is considered along with transient ZFS, where modulation is responsible for electron relaxation, provided molecular reorientation time is much longer than electron relaxation time (refs 51,52; incorporated by reference in their entireties).

C57BL/6N, wild-type mice were obtained. A 21G needle was used to inject annealed Gd(III)-PA solutions into the tibialis anterior muscle of each leg of each mouse. Approximately 4.5 μL were injected at a rate of 5 μL/min, while retracting the needle at a rate of 1 cm/min. This injection method allows for the PA solution to be introduced at a very slow rate, so that gelation may occur by in vivo divalent ions inside the needle tract. The injection and retraction was done using a modified NE-300 Just Infusion™ Syringe Pump. All animal studies were conducted at University of Illinois at Chicago and Northwestern University in accordance with the National Institutes of Health Guide for the Care and Use of Laboratory Animals and established institutional animal use and care protocols.

All images were acquired on a Bruker 7T Pharmascan MRI system using a 38 mm quadrature coil. Anatomical images were acquired with a fat-saturated multi-spin multi-echo (MSME) pulse sequence (TR/TE=800 ms/11.5 ms) with 5 slices of 1 mm thickness and two signal averages. The field of view was 4×4 cm with a 300×300 pixel matrix size. T₁ maps for the animal experiments were acquired using a rapid acquisition with relaxation enhancement (RARE)-based T₁ map protocol with repetition times 157, 200, 400, 800, 1200, 3000, and 4000 ms with one average and no fat saturation. T₂ acquisitions were taken using a fat-saturated MSME T₂ map sequence with repetition time 4000 ms and echo times 11.6, 23.3, 34.9, 46.5, 58.2, 69.8, 81.4, 93.1, 104.7, and 116.4 ms. Slice geometry parameters for T₁ and T₂ maps were the same as above. T₁ maps were generated with a saturation-recovery fit for each pixel using the Jim software package (Xinapse Systems, Colchester, UK).

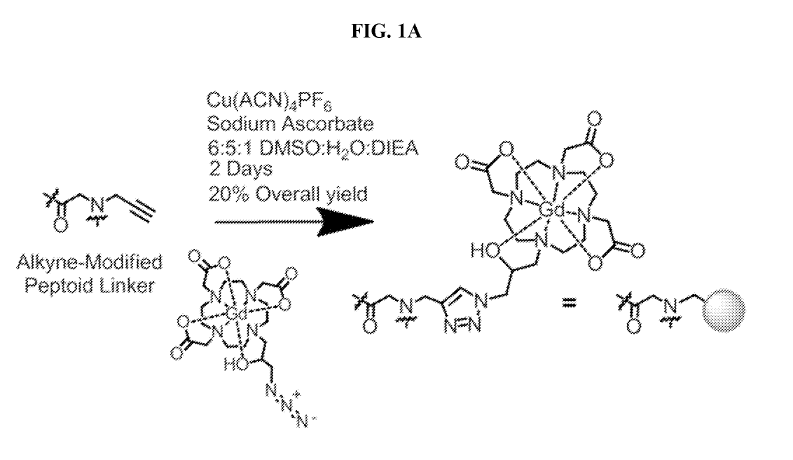

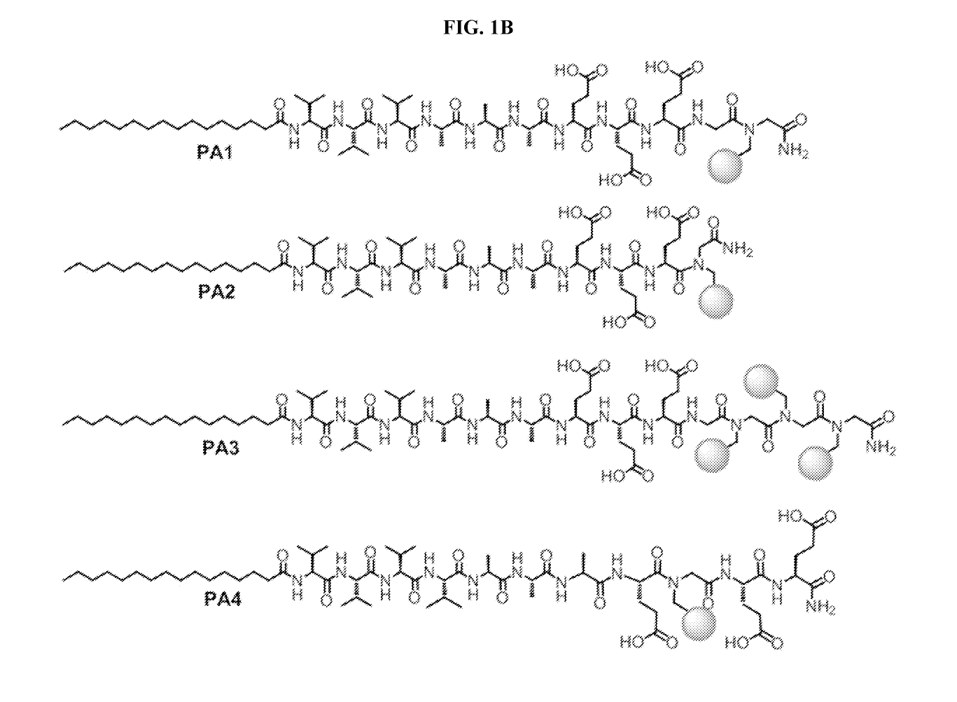

Peptide amphiphiles based on the V3A3 motif were chosen to promote one-dimensional assembly with strong tendency to form β-sheets (ref 38; incorporated by reference in its entirety). Three glutamic acid residues (e.g., acidic residues) were introduced in the peptide sequence to improve solubility and to promote strong interactions with divalent cations which can result in gel formation through charge screening. The basic PA structure explored here had a contrast agent conjugated at the C-terminus of the peptide PA1 with (PA1) or without a glycine linker (PA2). PA3 is similar to PA1 but it is conjugated with three chelates in order to investigate the potential benefits of trifunctional structures for imaging. In PA4, the Gd(III) contrast agent was moved closer to the fiber core while compensating for greater sterics with a stronger β-sheet (PA4) containing one additional valine residue. Peptide amphiphiles 1-4 were synthesized using solid-phase peptide synthesis, with peptoid linkers incorporated via established protocols (ref 9; incorporated by reference in its entirety). The peptoid residue linker provides a synthetically facile means of incorporating an alkyne functional handle. The Gd(III) macrocycle Gd(HPN3DO3A) was chosen for its chelate stability (ref 37,40,41; incorporated by reference in their entireties), and synthesized according to the method of Mastarone et al. (ref 42; incorporated by reference in its entirety) and was conjugated to the peptoids via click chemistry (FIG. 1A) to afford white powders in 20% overall yield after HPLC purification and lyophilization.

Small-angle X-ray scattering (SAXS) was used to provide data regarding nanostructure size, shape, and polydispersity in solution (FIG. 1B). Solutions were characterized at 1 mM in buffer after heating them to 80° C. and slowly cooling to room temperature. This procedure was used to impart ordering of filaments in solution to promote liquid crystallinity (ref 44; incorporated by reference in its entirety). The solutions became noticeably more viscous after annealing. After annealing, CaCl₂ was added (3.33 mM) to simulate calcium concentrations found in vivo. The effects of annealing followed by CaCl₂ addition was investigated.

To assess SAXS profiles for structural information, fits were applied based on a core-shell cylinder form factor. The slope of the Guiner region was approximately −1 for PA 1, 2 and 4, indicating one-dimensional, high-aspect-ratio structures. In contrast, the fit of PA 3 was consistent with much shorter fibers. The scattering curve can be fit to a core-shell cylinder with very short (˜10 nm) length. The deviation in slope observed at very low q in PA3 is likely due to aggregation for that compound. Annealing and calcium addition do not substantially change the SAXS form factors observed for PA1-4. However, the SAXS curve of PA1 reveals a small shift in the scattering minimum from q=0.06 to q=0.055 corresponding to a diameter change from 10.5 to 11.4 nanometers after thermal annealing, which indicates reorganization of the internal fiber structure. This increased radius may arise from thermal dehydration of the amino acids as the molecules adopt a more extended conformation.

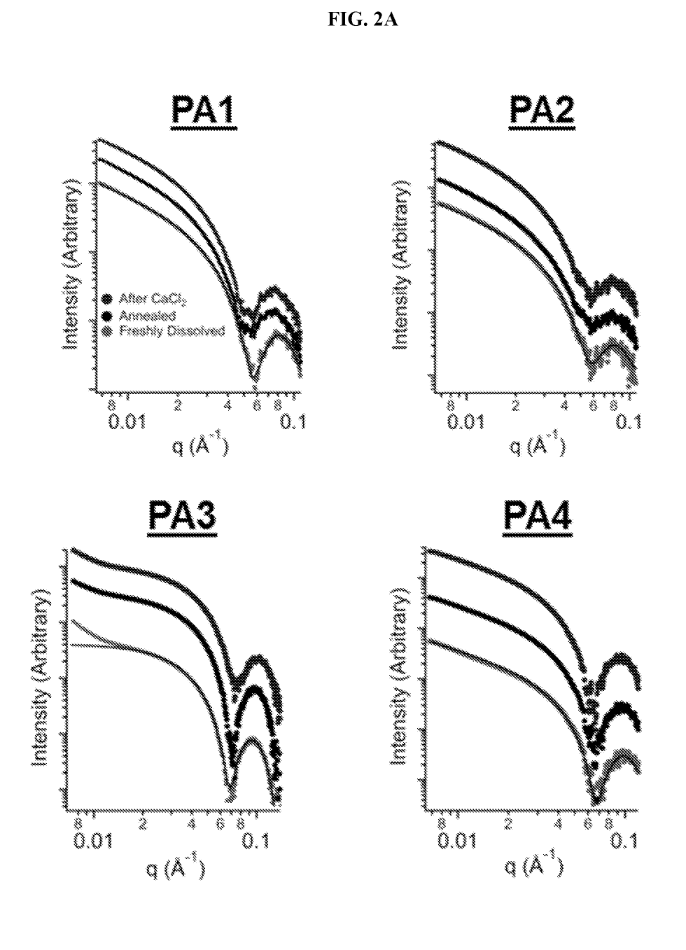

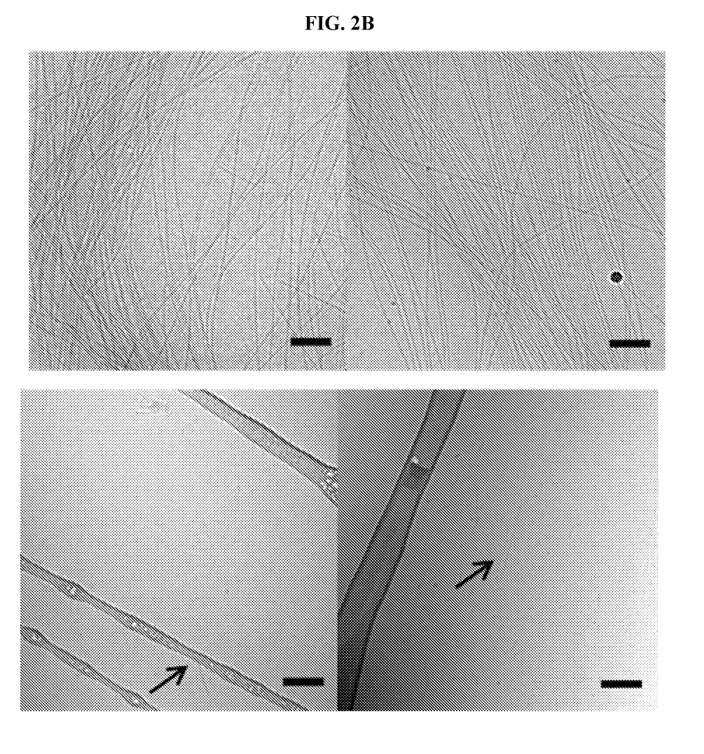

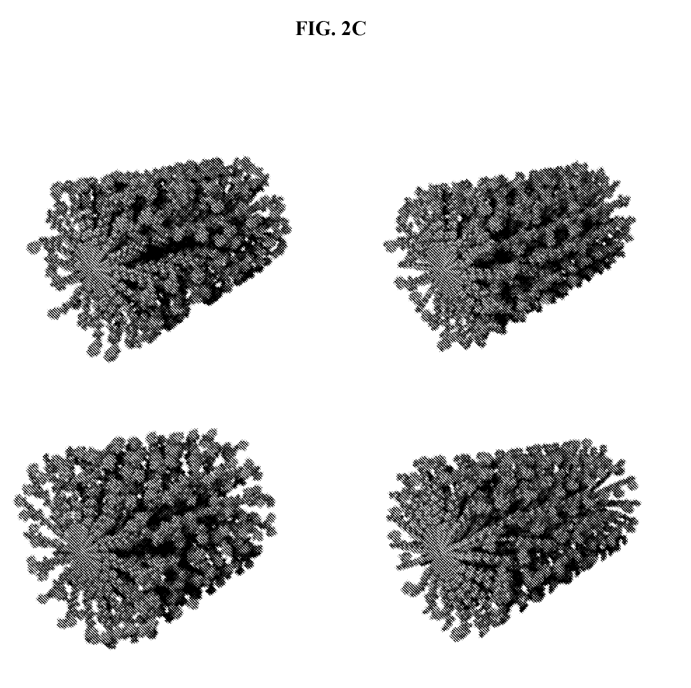

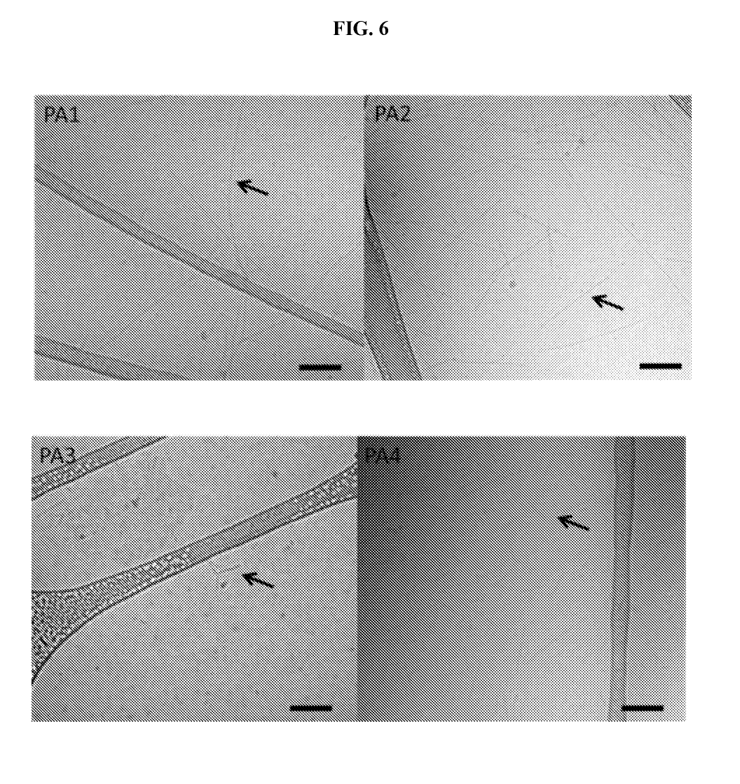

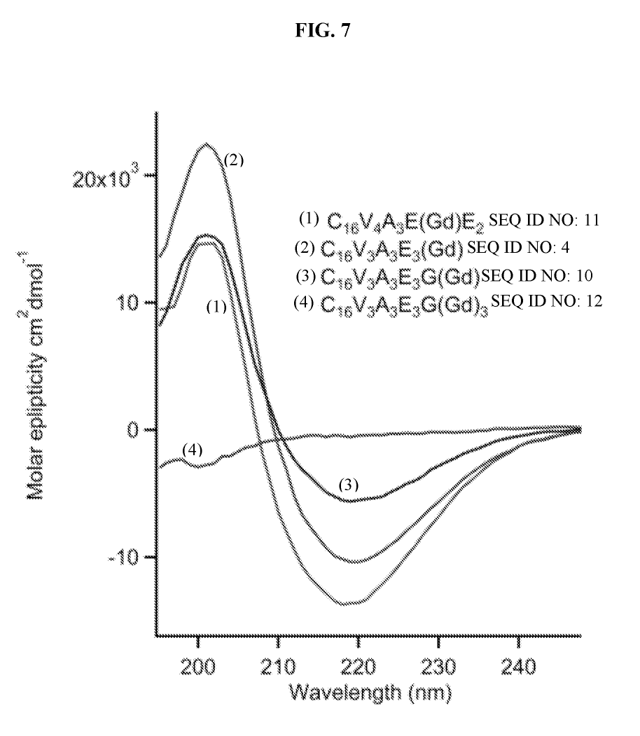

Cryogenic transmission electron microscopy (Cryo-TEM) was performed on all four PAs before and after thermal annealing (FIG. 6 and FIG. 2B respectively). After heating to 80° C. for 30 min and slowly cooling to room temperature, PA1-2 fibers appeared longer (several microns instead of hundreds of nanometers) and were more numerous. PA3-4 showed short fibers only a few hundred nanometers long before and after annealing. Despite an additional valine residue to strengthen β-sheets in assembled PA4, steric repulsion among the macrocycles on adjacent molecules likely suppresses long fiber formation. These results indicate that the bulky groups should be conjugated to the outermost residue of a short PA sequence to minimize disruption of the PA nanofiber morphology and retain high-aspect-ratio structures.

Circular dichroism (CD) spectroscopy was used to determine the secondary structure of the assembled PA molecules. The CD spectra indicated strong β-sheet character for all the assemblies except for PA 3, which exhibited a random coil structure FIG. 6, Table 4). The disruption of the β-sheet structure in PA 3 is likely due to the steric contribution of three bulky Gd(HP-DO3A) macrocycles. PA4 shows an intense β-sheet signal, as expected for a sequence with an additional valine residue due to this amino acid high propensity to form β-sheets.43 These CD results show a correlation between high-aspect-ratio fibers and β-sheet content for these conjugates.

Using a Bruker Minispect relaxometer operating at 1.41 T, T₁ and T₂ relaxivity values (r1 and r2) were determined for each agent (Table 1). At higher concentrations and long repetition times, T₂ relaxation is expected to dominate, generating negative contrast. At lower concentrations and short repetition times, T₁ relaxation is more likely to dominate, generating positive contrast. T₁ relaxivity of all supramolecular nanostructures shows substantial improvement when compared to commercial agents (r1 ˜4 mM-1s−1). These results show that PAs 1-4 are potentially useful to label PA implants for fate-mapping. Thermal annealing followed by addition of CaCl₂ caused complex changes in the observed agent relaxivity (Table 1). PA1 showed an increase in relaxivity upon thermal annealing, as expected for a macrocycle more accessible to bulk solvent. This increased accessibility is consistent with a more extended molecular structure as indicated by SAXS. In contrast, PA2 shows no change in relaxivity with annealing, but relaxivity increases upon CaCl₂ addition to the annealed solutions. PA3-4, which did not produce high aspect-ratio fibers, showed little change in relaxivity when annealed or when Ca²⁺ ions were added for charge screening (possibly electrostatic binding among nanofibers). Based on these results, increased relaxivity on annealing and is expected for peptide amphiphiles exhibiting β-sheet character that generate high aspect-ratio fiber nanostructures.

TABLE 1
Summary of PA T₁ and T₂ relaxivities as a function of condition.
All measurements were obtained at 1.41 tesla.

| | H₂O | | Tris Buffer (pH 7.4) | | After Thermal Annealing | | Addition of CaCl₂ | |
|---|---|---|---|---|---|---|---|---|
| | r₁ | r₂ | r₁ | r₂ | r₁ | r₂ | r₁ | r₂ |
| PA1 | 16.5 ± 0.5 | 34 ± 1 | 16 ± 2 | 30 ± 4 | 19.0 ± 0.6 | 37 ± 1 | 18.4 ± 0.2 | 40.0 ± 0.1 |
| PA2 | 17.3 ± 0.8 | 39 ± 2 | 16.7 ± 0.2 | 32.2 ± 0.5 | 16.9 ± 0.2 | 28.6 ± 0.6 | 21.7 ± 0.1 | 45 ± 2 |
| PA3 | 16.4 ± 0.6 | 30 ± 2 | 15.8 ± 0.5 | 25.3 ± 0.2 | 16.4 ± 0.6 | 25.2 ± 0.2 | 16.5 ± 0.8 | 25 ± 1 |
| PA4 | 17 ± 1 | 31 ± 2 | 15.6 ± 0.1 | 25.3 ± 0.2 | 16.0 ± 0.1 | 25.2 ± 0.2 | 18 ± 1 | 25 ± 1 |

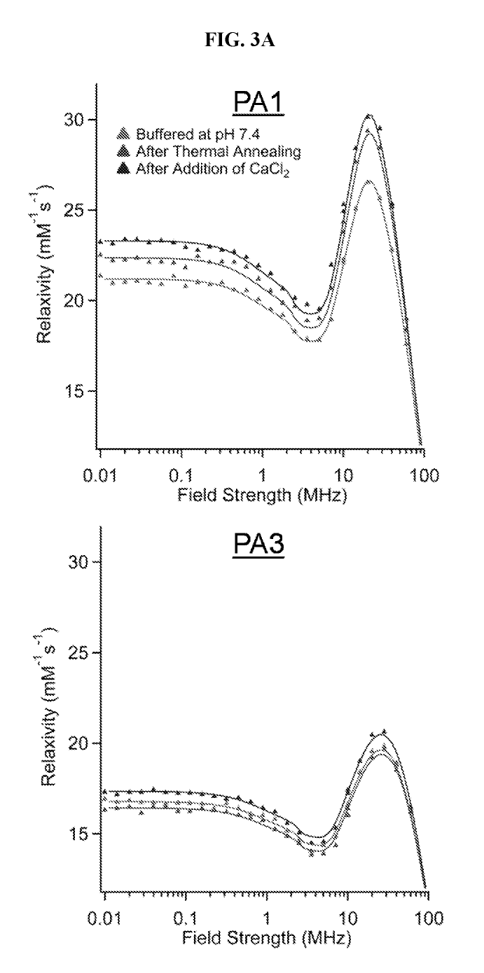

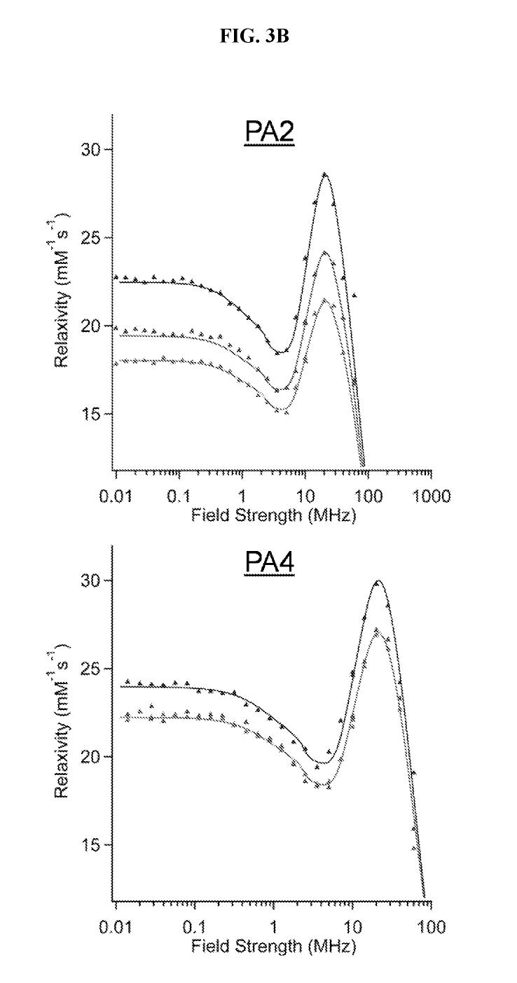

In order to establish which parameter was dominant in relaxivity changes, NMRD profiles of each PA were measured. NMRD profiles for all four PAs at 25° and 37° C. are shown in FIG. 3 with parameter fits obtained using the “modified Florence” approach (refs. 44-46;1 incorporated by reference in their entireties). In the fits, a Lipari-Szabo order parameter S² was included to consider the effect of local motions which may superimpose to the global motion of the nanostructures. A smaller S² value indicates a less restricted local mobility (e.g., a more flexible linkage between fiber and macrocycle). PA3 shows a substantially larger local motional freedom, consistent with its random coil secondary structure. The NMRD fits show τm is primarily responsible for the variation in relaxivity observed upon annealing and Ca2+ cross-linking (Table 2). It is contemplated that annealing and calcium addition change macrocycle presentation on the fiber surface, extending them from the structure with increased peptide packing density and facilitating faster water exchange. The same changes in τM are not observed for PAs that do not form high-aspect-ratio assemblies (PA3-4), implying that the packing of the PA1-2 assemblies is key to this τM effect.

TABLE 2
Summary of best-fit values of key parameters obtained from
fits at 37° C.

| | τm (ns) | | | | |
|---|---|---|---|---|---|
| | Buffered | Annealed | Ca²⁺ | S² | τ_{local} (ns) |
| PA1 | 465 | 400 | 380 | 0.25 | 4 |
| PA2 | 640 | 540 | 410 | 0.3 | 4 |
| PA3 | 690 | 700 | 650 | 0.0 | 4 |
| PA4 | 480 | 480 | 420 | 0.25 | 5.5 |

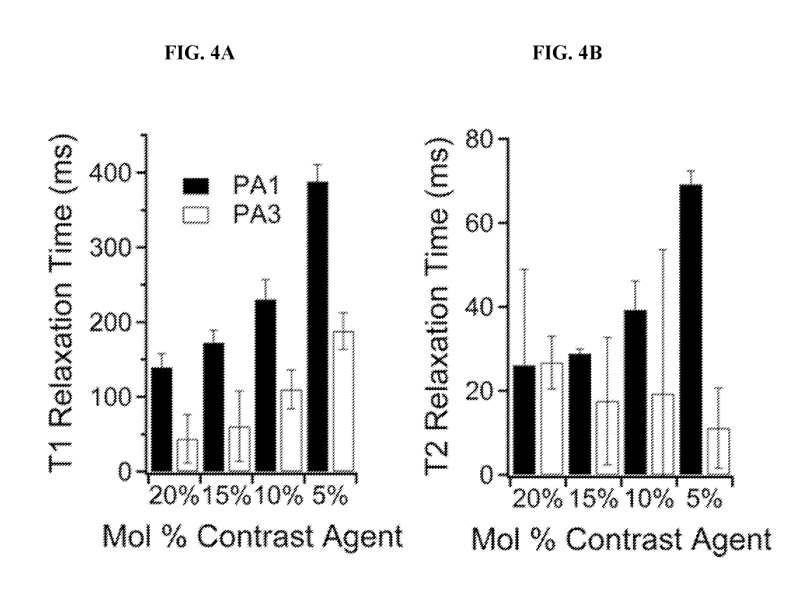

PA1 produced high aspect-ratio nanostructures and PA3, with its three appended contrast agents, does not form high-aspect-ratio nanostructures. These two PAs were chosen for testing to follow in vivo degradation. To determine optimum contrast agent loading, PA1 and PA3 were mixed in varied ratios with C₁₆V₃A₃E₃-NH₂ PA. The total concentration of unlabeled PA and Gd(III)-PA was kept constant at 1.3 mM, and measurements were obtained using T₁ and T₂ mapping sequences at 7 T. T₁ showed an inverse relationship versus gadolinium concentration, as expected for a homogeneous agent in solution (FIG. 4A) (ref 47; incorporated by reference in its entirety). This indicates that relaxivity is independent of Gd(III)-PA to unlabeled PA ratio. At 10 mol % PA, T₁ relaxation time was sufficiently fast (100-250 ms for both conjugates) to generate large contrast.

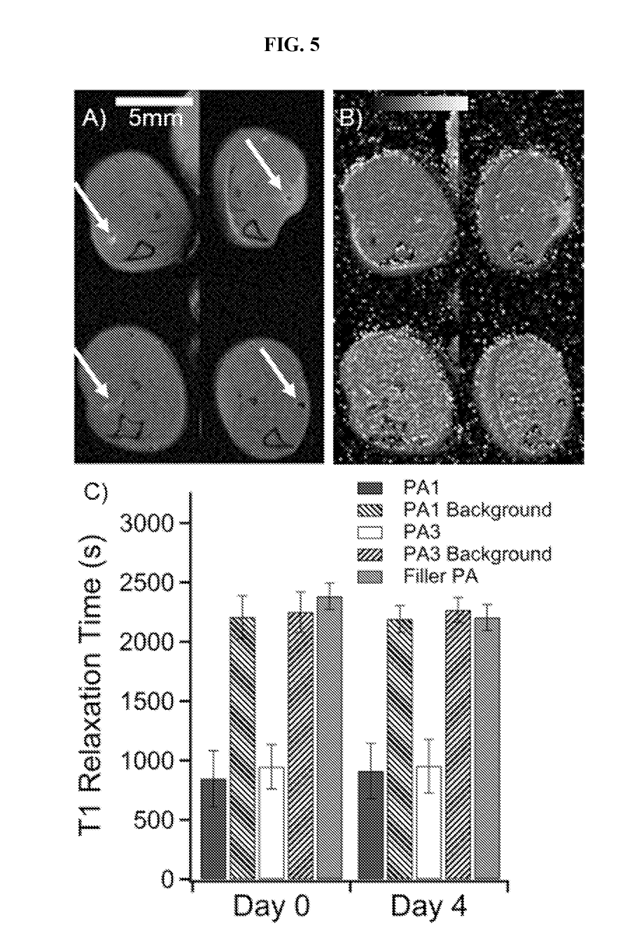

To measure degradation in vivo, six wild-type mice received approximately 4 μL injections of PA gels at 1 wt % loaded with 10 mol % Gd(III)-PA relative to C₁₆V₃A₃E₃-NH₂. The injections were applied with a modified syringe pump designed to inject while withdrawing the needle to produce a gelled track of PA in situ. Injection volume varied because track length varied slightly with mouse size and small variations in each injection geometry. The tract geometry was chosen to give accurate T₁ determinations because imaging slice thickness could be greater without volume averaging effects. A total of five legs were injected with PA1, four legs with PA3, and three with 100% C₁₆V₃A₃E₃-NH₂ as a control. Mice were imaged at day 0 immediately after injections and again at day 4. The Gd(III)-PA was clearly visible in all cases (FIG. 5). PA1 produced T1-weighted positive contrast while PA4 produced T2-weighted negative contrast. After day 4, the mice were sacrificed and the leg muscles were subjected to ICP-MS analysis to measure Gd(III) retention. ICP-MS revealed that 62±8% of PA1 and 54±9% of PA3 remained in the mouse leg after four days. The T₁ relaxation time of regions of interest did not appreciably change after four days as shown in FIG. 5C. Stable T₁ times indicate that PA concentration did not change substantially over the four day period. These ICP-MS and T1 map results show Gd(III)-PAs are useful for following PA gel biodegradation over time.

### Example 2

### Peptide-Peptoid Synthesis

Rink amide MBHA resin, HBTU, Oxymapure, and Fmoc-amino acids were obtained from Anaspec Corporation (Fremont, Calif.). DMF, DCM, and NMP were obtained from VWR International. DMF was dried over activated alumina column prior to use. N,N′-Diisopropylethlyamine (DIEA), acetic anhydride, bromoacetic acid, and propargyl amine were obtained from Sigma-Aldrich. Unless otherwise noted, all other reagents were obtained from Sigma and used without further purification.

Peptoid linker synthesis was conducted using the procedure of Zuckerman with the following modifications (ref 53; incorporated by reference in its entirety). The synthesis was conducted at a 0.5 mmol scale in a fritted shaker vessel. The resin was deprotected with piperidine (25 mL, 30% in DMF) for 10 minutes. A solution of bromoacetic acid (10 mL, 0.6 M) in dry DMF and 1.05 mL of diisopropylcarbodiimide, was allowed to react for 1 minute and was added to the resin. After 30 minutes, a second addition of bromoacetic acid was performed and the solution shaken for an additional 45 minutes. When ninhydrin confirmed successful addition, the resin was washed with DMF and DCM and a solution of propargylamine (5 mL, 1.2 M) was added to the resin and agitated overnight. Applying a few drops a solution of chloranil (1% in DMF) and solution of acetaldehyde (1% DMF) to a few of the resin beads produced a green color—indicating the presence of a secondary amine.

Coupling peptoid linker to amino acid was conducted using 7.9 equivalents of HBTU to 8 equivalents fmoc-amino acid (glycine or glutamic acid) and 12 equivalents of DIEA. All other amino acids were added using a CS Bio Co (Model CS136) peptide synthesizer. For amino acid coupling, 4 equivalents of amino acid, 4 equivalents of Oxymapure, and 4 equivalents of DIC were used. Between each coupling step, a solution of 10% acetic anhydride in DCM was added to cap unreacted amines (ref 54; incorporated by reference in its entirety).

Synthesis of the azide-functionalized macrocycle was conducted as previously described (ref 3; incorporated by reference in its entirety). To couple the macrocycle and peptide amphiphile, 10 mg of sodium ascorbate and 2 mg of Tris((1-benzyl-1H-1,2,3-triazol-4-yl)methyl)amine TBTA was added to a round bottom flask. Two equivalents of azide to crude PA were added to the flask in minimum volumes of Millipore water and DMSO respectively. The reaction proceeded over 24 hours at 50° C. in a mixture of 6:5:1 DMSO/H2O/DIEA. DIEA was removed by rotary evaporation and the DMSO/water mixture removed via lyophilization. The resulting orange-yellow powder was purified using reverse-phase HPLC in H2O/MeCN with 0.1% NH4OH in the mobile phase.

### Quantification of Gd(III) Concentration Using Inductively Coupled Mass Spectrometry (ICP-MS)

All concentrations used in relaxivity determination were obtained using ICP-MS. Samples were digested using ACS reagent grade nitric acid (70%) for a least 2 hours at 70° C. Samples were diluted in filtered, deionized water. A standard containing the elements Bi, Ho, In, Li, Sc, Tb, and Y was added to a final concentration of 5 ng/mL (Inorganic Ventures, Christiansburg, Va., USA). Final nitric acid concentration was 3%. The instrument was calibrated using a serial dilution of Gd(III) standards (Inorganic Ventures, Christiansburg, Va., USA) with nitric acid and internal standard concentrations identical to the samples. Calibration was conducted with 1.000, 5.000, 10.00, 20.00, 50.00, 100.0, and 200.0 ng/mL Gd(III) standards.

For Gd(III) analysis, mouse leg muscles were excised and digested in 1 mL of nitric acid per gram of tissue in Teflon tubes. A Milestone EthosEZ microwave digestion system (Shelton, Conn., USA) was used to digest the samples, ramping to 120° C. over 30 minutes, then holding at 120° C. for an additional 30 minutes. Digest was diluted and measured using the same conditions as above.

All measurements used a Thermo X series II ICP-MS (Thermo Fisher Scientific, Waltham, Mass., USA) operating with an ESI SC-2 autosampler (Omaha, Nebr., USA). Each acquisition consisted of one survey scan, utilizing 10 sweeps, and 3 peak jumping measurements of 100 sweeps each. The two most common isotopes of Gd, 157Gd and 158Gd, were measured. 115In and 165Ho were measured as internal standards.

### Purification

Reverse-phase, preparative HPLC was employed to purify the conjugates. A Varian Prostar 210 HPLC system supplied a 2% to 100% ACN to water gradient to a Phenomenex Gemini C18 column (5 μm particle size). To each solution was added 0.1% NH4OH to maintain peptide solubility. Fractions were analyzed for product content on an Agilent 6510 Q-TOF MS, then recombined. ACN was removed with rotary evaporation, and the water then removed via lyophilization. The white, fluffy powders were stored dry at −20° C.

TABLE 3
HPLC methods for the purification of each contrast agent PA.

| PA1 | | PA2 | | PA3 | | PA4 | |
|---|---|---|---|---|---|---|---|
| Elution Time (min) | | Elution Time (min) | | Elution Time (min) | | Elution Time (min) | |
| 22.5 | | 22 | | 12.5 | | 1.5 | |
| Method | | Method | | Method | | Method | |
| Time | % | Time | % | Time | % | Time | % |
| (min) | Acetonitrile | (min) | Acetonitrile | (min) | Acetonitrile | (min) | Acetonitrile |
| 0 | 2 | 0 | 2 | 0 | 2 | 0 | 2 |
| 5 | 2 | 5 | 2 | 2 | 2 | 5 | 2 |
| 13 | 18 | 12 | 20 | 4.4 | 14 | 13 | 26 |
| 27 | 25 | 39 | 60 | 6.6 | 15.9 | 27 | 42 |
| 38 | 100 | 43 | 100 | 19.7 | 17 | 30 | 70 |
| 42 | 100 | 48 | 100 | 30 | 80 | 38 | 100 |
| | | | | 40 | 100 | 42 | 100 |

### Analytical HPLC

Analytical HPLC was performed using Varian Prostar 500 HPLC equipped with a Varian 380LC ELSD system to generate the traces. An X-Bridge C18 column with dimensions 4.6×150 mm and 5 μm pore size was used. NH4OH solution at pH 10.3 was used as the polar mobile phase and acetonitrile as the nonpolar. A flow rate of 1 mL/min was used in all cases. Analytical traces are shown below, with peak elution times noted in the captions. Solutions were prepared at approximately 1 mM and injected using the following method:

TABLE 4
Analytical HPLC method used to verify agent purity.

| Time (min) | % ACN |
|---|---|
| 0 | 5 |
| 8 | 5 |
| 55 | 100 |
| 65 | 100 |
| 75 | 5 |
| 80 | 5 |

### MALDI-MS

A Bruker AutoFLex III high Resolution MALDI-TOF mass spectrometer was used to collect MALDI-MS spectra. Bruker Daltonics Prespotted AnchorChip 96 MALDI Targets were used as targets, prespotted with HCA.

### TEM

For Cryogenic transmission electron microscopy (Cryo-TEM), two solutions of 1 mmol of each compound (PA1-4) were prepared at pH 7.4. Unannealed specimens were aged overnight prior to imaging. For imaging annealed specimens, the solution was annealed at 80° C. for 30 minutes before being allowed to cool in the bath slowly overnight. Imaging was conducted on a JEOL 1230 microscope, operating at 100 kV. In a humidity and temperature controlled Vitrobot VI, 5 μL of sample was deposited on a suspended grid, blotted, and plunged into liquid ethane to produce vitreous ice. Samples were transported in liquid nitrogen, and transferred to a Gatan 626 cryo holder, cooled with liquid nitrogen. The sample remained at liquid nitrogen temperatures during imaging for all samples.

### Circular Dichroism

CD spectroscopy was performed with the same solutions used in cryo-EM, except they had been diluted 10× in Tris buffer to a final concentration of 0.1 mM. A J-715 Jasco Circular Dichroism spectrometer was used for these measurements. All measurements were taken at 37° C. Red shifts were calculated from the minima of the negative sigmoid peak characteristic of β-sheet structures. Each spectrum is the average of three scans.

### Relaxation Measurements

T₁ Measurements were performed at 1.41 T with a Bruker Minispect mq60 magnet, using an inversion recovery sequence. T₂ measurements were acquired using a Carr-Purcell-Meiboom-Gill (CPMG) pulse sequence. PAs were weighed out and dissolved in Millipore water to form 1 mg/mL solutions, and then these solutions were serial diluted to 0.5, 0.25, 0.125, 0.05, and 0.01 mg/mL concentrations. For each concentration, T₁ and T₂ were measured. These solution concentrations were then measured using ICP-MS for relaxivity determination. Samples were lyophilized and resuspended in 50 mM Tris and 150 mM NaCl buffer. These were then adjusted using small volumes of 0.1 M NaOH to reach pH 7.4. T1 and ICP-MS measurements were then taken of these solutions. The relaxometer tubes were heated at 80° C. for 30 min in a water bath. The bath was allowed to come to room temperature over several hours. T₁ times were measured. Finally, 100 μL of 20 mM CaCl2 solution was added to each tube. The tubes were vortex-mixed and allowed to stand for 20 minutes. ICP-MS and T₁ measurements were conducted once more.

### NMRD Profiles

The ¹H NMRD profiles were analyzed assuming one water molecule regularly coordinated to the Gd(III) ion in the presence of freely diffusing outer-sphere water. The contribution of the coordinated water protons (R_{1M}) to the relaxation of bulk water protons (R1) largely depends on the τM of coordinated water if τM>R_{1M}, as expected for Gd(HP-DO3A), according to the following relationship:

R₁=f_{M}(1/R_{1M}+τM)⁻¹+Rₒₛ

where f^{M} is the mole fraction of ligand nuclei in bound positions and Rₒₛ is the outer-sphere contribution. R_{1M} is calculated by considering the simultaneous presence of both static (D) and transient (Δt) ZFS, as needed for Gd(III) complexes, through the “modified Florence” NMRD program, 4-6 assuming slow rotation, i.e. the molecular tumbling time τR is much longer than the electron relaxation time Tₗₑ. The PA fiber's micron-scale size assures that TR could be fixed to a large, constant value (≥100 ns) in the calculation of the relaxation profile.

The ¹H NMRD profiles of PA 1 were repeated at both temperatures using the same Δt value of 0.0142 cm-1, D=0.046 cm-1, θ=42° (angle between the z-axis of the ZFS tensor and the metal-water direction) and a correlation time for electron relaxation τV=24 ps. A distance of closest approach of diffusing water molecules (d) of 7-10 Angstroms should be set, i.e. somewhat larger than usually found in isolated Gd(III) chelates (3.5-4 Å). The proximity of the peptide chains in the nanofibers precludes water approach to the Gd(III) chelates from many directions, thus decreasing the outer-sphere contribution Ros. The best-fit values of the other parameters are reported in Table 2.

A good fit of the high field region of the profiles could be obtained only by assuming the presence of local mobility, with a correlation time T_{local}, considered together with the global reorientation time τ_{R} using the Lipari-Szabo model free formalism (refs. 7, 8; incorporated by reference in their entireties). In this model, the order parameter S² tunes the degree of spatial restriction of the local motions, from totally free (S²=0) to quenched (S²=1).

The 1H NMRD profiles of the other compounds could then be reproduced by fitting only the parameters τ_{M}, S², and τ_{local}, with the constraint that S² should be the same at the two measurement temperatures (25 and 37° C.). Finally, the 1H NMRD profiles of the compounds after thermal annealing and in the presence of Ca(II) could be reproduced using the same set of parameters producing the profiles of the corresponding compound acquired without Ca(II), at the same temperature, and adjusting only the value of τM.

### Small Angle X-Ray Scattering

SAXS measurement was performed at beam line 5ID-D, in the DuPont-Northwestern-Dow Collaborative Access team (DND-CAT) Synchrotron Research Center at the Advanced Photon Source, Argonne National Laboratory. The wavelength, selected with double-crystal monochrometer, was 0.83 Å. A MAR CCD detector was positioned 245 cm behind the sample. Samples were loaded into 1.5 mm quartz capillaries and irradiated. All compounds (PAs 1-4) were prepared at 0.1 mM in solutions of Tris buffered saline (50 mM Tris 150 mM NaCl).

### Example 3

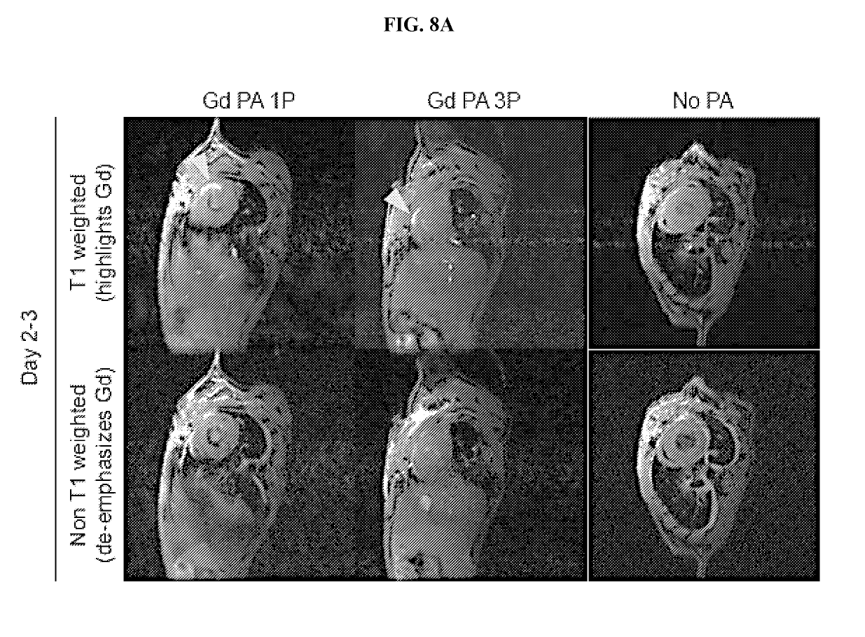

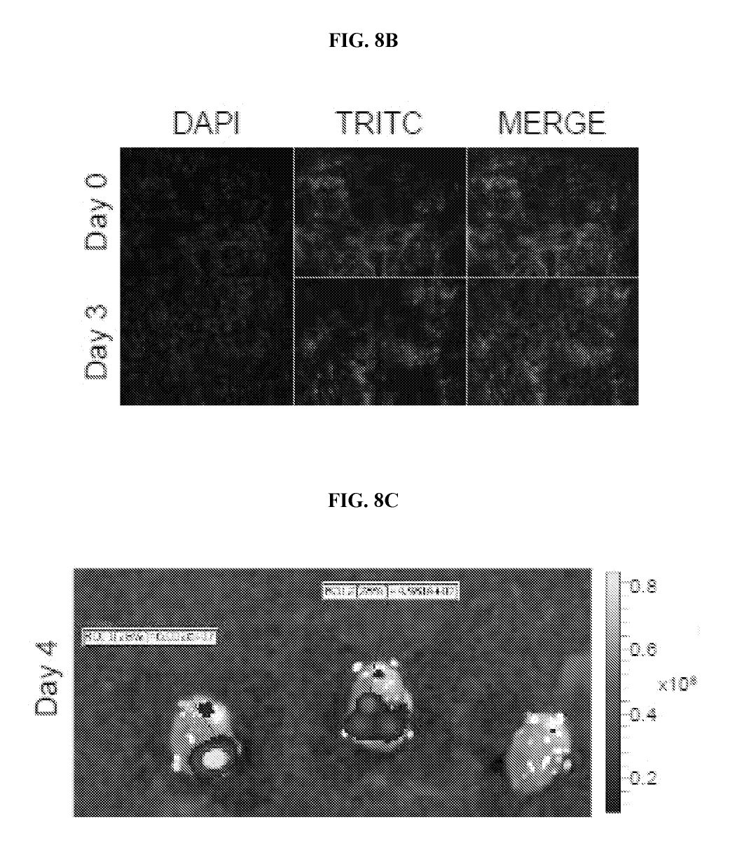

In order to investigate the interaction between cells, PA, and tissue in vivo, aspects of the production and delivery of PA molecules that chelate Gadolinium ions to be used as contrast agents in magnetic resonance imaging (MRI) have been optimized. Experiments were conducted during development of embodiments of the present invention to observe the PA nanofibers in the heart muscle by MRI when as little as 10% of the overall material was doped with the Gadolinium-containing PA (FIG. 8A). A fluorescently-labeled PA was used to confirm the presence of the PA in the heart by looking at histological sections (FIG. 8B) or by using an In Vivo Imaging System (IVIS) in whole hearts (FIG. 8C). This allows for correlation of the presence of the injected PA nanofibers-cells/proteins with the biological outcome of the treatment.

### REFERENCES

The following references, some of which are referenced in the text above by number, are herein incorporated by reference in their entireties:
(1) Stupp, S. I.; Palmer, L. C. Supramolecular Chemistry and Self-Assembly in Organic Materials Design. Chem. Mater. 2013, 26, 507-518.
(2) Webber, M. J.; Berns, E. J.; Stupp, S. I. Supramolecular Nanofibers of Peptide Amphiphiles for Medicine. Isr. J. Chem. 2013, 53, 530-554.
(3) Palmer, L. C.; Stupp, S. I. Molecular Self-Assembly into One-Dimensional Nanostructures. Accounts Chem. Res. 2008, 41, 1674-1684.
(4) Aida, T.; Meijer, E. W.; Stupp, S. I. Functional Supramolecular Polymers. Science 2012, 335, 813-817.
(5) Shah, R. N.; Shah, N. A.; Del Rosario Lim, M. M.; Hsieh, C.; Nuber, G.; Stupp, S. I. Supramolecular Design of Self-Assembling Nanofibers for Cartilage Regeneration. P. Natl. Acad. Sci. USA 2010, 107, 3293-3298.
(6) Lee, S. S.; Huang, B. J.; Kaltz, S. R.; Sur, S.; Newcomb, C. J.; Stock, S. R.; Shah, R. N.; Stupp, S. I. Bone Regeneration with Low Dose Bmp-2 Amplified by Biomimetic Supramolecular Nanofibers within Collagen Scaffolds. Biomaterials 2012, 452-459.
(7) Webber, M. J.; Tongers, J.; Newcomb, C. J.; Marquardt, K. T.; Bauersachs, J.; Losordo, D. W.; Stupp, S. I. Supramolecular Nanostructures That Mimic Vegf as a Strategy for Ischemic Tissue Repair. P. Natl. Acad. Sci. USA 2011, 108, 13438-13443.
(8) Webber, M. J.; Han, X. Q.; Murthy, S. N. P.; Rajangam, K.; Stupp, S. I.; Lomasney, J. W. Capturing the Stem Cell Paracrine Effect Using Heparin-Presenting Nanofibres to Treat Cardiovascular Diseases. J Tissue Eng Regen M 2010, 4, 600-610.
(9) Silva, G. A.; Czeisler, C.; Niece, K. L.; Beniash, E.; Harrington, D. A.; Kessler, J. A.; Stupp, S. I. Selective Differentiation of Neural Progenitor Cells by High-Epitope Density Nanofibers. Science 2004, 303, 1352-1355.
(10) Beniash, E.; Hartgerink, J. D.; Storrie, H.; Stendahl, J. C.; Stupp, S. I. Self-Assembling Peptide Amphiphile Nanofiber Matrices for Cell Entrapment. Acta Biomater. 2005, 1, 387-397.
(11) Hartgerink, J. D.; Beniash, E.; Stupp, S. I. Self-Assembly and Mineralization of Peptide-Amphiphile Nanofibers. Science 2001, 294, 1684-1688.
(12) Webber, M. J.; Kessler, J. A.; Stupp, S. I. Emerging Peptide Nanomedicine to Regenerate Tissues and Organs. J. Intern. Med. 2010, 267, 71-88.
(13) Pashuck, E. T.; Cui, H. G.; Stupp, S. I. Tuning Supramolecular Rigidity of Peptide Fibers through Molecular Structure. J. Am. Chem. Soc. 2010, 132, 6041-6046.
(14) Pashuck, E. T.; Stupp, S. I. Direct Observation of Morphological Tranformation from Twisted Ribbons into Helical Ribbons. J. Am. Chem. Soc. 2010, 132, 8819-8821.
(15) Zhang, S. M.; Greenfield, M. A.; Mata, A.; Palmer, L. C.; Bitton, R.; Mantei, J. R.; Aparicio, C.; de la Cruz, M. O.; Stupp, S. I. A Self-Assembly Pathway to Aligned Monodomain Gels. Nat. Mater. 2010, 9, 594-601.
(16) Ortony, J. H. N., C.; Matson, J. B; Palmer, L. C.; Doan, P. E.; Hoffman, B. M.; Stupp, S. I. Internal Dynamics of a Supramolecular Nanofiber. Nat. Mater. 2014, In Press.
(17) Shah, R. N.; Shah, N. A.; Lim, M. M. D.; Hsieh, C.; Nuber, G.; Stupp, S. I. Supramolecular Design of Self-Assembling Nanofibers for Cartilage Regeneration. P. Natl Acad. Sci. USA 2010, 107, 3293-3298.
(18) Storrie, H.; Guler, M. O.; Abu-Amara, S. N.; Volberg, T.; Rao, M.; Geiger, B.; Stupp, S. I. Supramolecular Crafting of Cell Adhesion. Biomaterials 2007, 28, 4608-4618.
(19) Webber, M. J.; Tongers, J.; Renault, M. A.; Roncalli, J. G.; Losordo, D. W.; Stupp, S. I. Development of Bioactive Peptide Amphiphiles for Therapeutic Cell Delivery. Acta Biomater. 2010, 6, 3-11.
(20) Appel, A. A.; Anastasio, M. A.; Larson, J. C.; Brey, E. M. Imaging Challenges in Biomaterials and Tissue Engineering. Biomaterials 2013, 34, 6615-6630.
(21) Karfeld-Sulzer, L. S.; Waters, E. A.; Davis, N. E.; Meade, T. J.; Barron, A. E. Multivalent Protein Polymer Mn Contrast Agents: Controlling Relaxivity Via Modulation of Amino Acid Sequence. Biomacromolecules 2010, 11, 1429-1436.
(22) Karfeld-Sulzer, L. S.; Waters, E. A.; Kohlmeir, E. K.; Kissler, H.; Zhang, X.; Kaufman, D. B.; Barron, A. E.; Meade, T. J. Protein Polymer Mri Contrast Agents: Longitudinal Analysis of Biomaterials in Vivo. Magn. Reson. Med. 2011, 65, 220-228.
(23) Frullano, L.; Meade, T. Multimodal Mri Contrast Agents. JBIC Journal of Biological Inorganic Chemistry 2007, 12, 939-949.
(24) Hung, A. H.; Duch, M. C.; Parigi, G.; Rotz, M. W.; Manus, L. M.; Mastarone, D. J.; Dam, K. T.; Gits, C. C.; MacRenaris, K. W.; Luchinat, C.; Hersam, M. C.; Meade, T. J. Mechanisms of Gadographene-Mediated Proton Spin Relaxation. The Journal of Physical Chemistry C 2013, 117, 16263-16273.
(25) Matosziuk, L. M.; Leibowitz, J. H.; Heffern, M. C.; MacRenaris, K. W.; Ratner, M. A.; Meade, T. J. Structural Optimization of Zn(Ii)-Activated Magnetic Resonance Imaging Probes. Inorg. Chem. 2013, 52, 12250-12261.
(26) Caravan, P.; Ellison, J. J.; McMurry, T. J.; Lauffer, R. B. Gadolinium(Iii) Chelates as Mri Contrast Agents: Structure, Dynamics, and Applications. Chem. Rev. 1999, 99, 2293-2352.
(27) Bull, S. R.; Guler, M. O.; Bras, R. E.; Venkatasubramanian, P. N.; Stupp, S. I.; Meade, T. J. Magnetic Resonance Imaging of Self-Assembled Biomaterial Scaffolds. Bioconjug. Chem. 2005, 16, 1343-1348.
(28) Bull, S. R.; Guler, M. O.; Bras, R. E.; Meade, T. J.; Stupp, S. I. Self-Assembled Peptide Amphiphile Nanofibers Conjugated to Mri Contrast Agents. Nano Lett. 2005, 5, 1-4.
(29) Ghosh, A.; Haverick, M.; Stump, K.; Yang, X.; Tweedle, M. F.; Goldberger, J. E. Fine-Tuning the Ph Trigger of Self-Assembly. J. Am. Chem. Soc. 2012, 134, 3647-3650.
(30) Caravan, P.; Parigi, G.; Chasse, J. M.; Cloutier, N. J.; Ellison, J. J.; Lauffer, R. B.; Luchinat, C.; McDermid, S. A.; Spiller, M.; McMurry, T. J. Albumin Binding, Relaxivity, and Water Exchange Kinetics of the Diastereoisomers of Ms-325, a Gadolinium(Iii)-Based Magnetic Resonance Angiography Contrast Agent. Inorg. Chem. 2007, 46, 6632-6639.
(31) Zech, S. G.; Sun, W. C.; Jacques, V.; Caravan, P.; Astashkin, A. V.; Raitsimring, A. M. Probing the Water Coordination of Protein-Targeted Mri Contrast Agents by Pulsed Endor Spectroscopy. Chemphyschem. 2005, 6, 2570-2577.
(32) Zech, S. G.; Eldredge, H. B.; Lowe, M. P.; Caravan, P. Protein Binding to Lanthanide(Iii) Complexes Can Reduce the Water Exchange Rate at the Lanthanide. Inorg. Chem. 2007, 46, 3576-3584.
(33) Manus, L. M.; Mastarone, D. J.; Waters, E. A.; Zhang, X. Q.; Schultz-Sikma, E. A.; Macrenaris, K. W.; Ho, D.; Meade, T. J. Gd(Iii)-Nanodiamond Conjugates for Mri Contrast Enhancement. Nano Lett. 2010, 10, 484-489.
(34) Major, J. L.; Parigi, G.; Luchinat, C.; Meade, T. J. The Synthesis and in Vitro Testing of a Zinc-Activated Mri Contrast Agent. P. Natl Acad. Sci. USA 2007, 104, 13881-13886.
(35) Li, W. H.; Parigi, G.; Fragai, M.; Luchinat, C.; Meade, T. J. Mechanistic Studies of a Calcium-Dependent Mn Contrast Agent. Inorg. Chem. 2002, 41, 4018-4024.
(36) Song, Y.; Xu, X.; MacRenaris, K. W.; Zhang, X. Q.; Mirkin, C. A.; Meade, T. J. Multimodal Gadolinium-Enriched DNA-Gold Nanoparticle Conjugates for Cellular Imaging. Angew. Chem. Int. Edit. 2009, 48, 9143-9147.
(37) Tweedle, M. F. The Prohance Story: The Making of a Novel Mri Contrast Agent. Eur Radiol 1997, 7, S225-S230.
(38) Newcomb, C. J.; Sur, S.; Ortony, J. H.; Lee, O.-S.; Matson, J. B.; Boekhoven, J.; Yu, J. M.; Schatz, G. C.; Stupp, S. I. Cell Death Versus Cell Survival Instructed by Supramolecular Cohesion of Nanostructures. Nat Commun 2014, 5.
(39) Nam, K. T.; Shelby, S. A.; Choi, P. H.; Marciel, A. B.; Chen, R.; Tan, L.; Chu, T. K.; Mesch, R. A.; Lee, B. C.; Connolly, M. D.; Kisielowski, C.; Zuckermann, R. N. Free-Floating Ultrathin Two-Dimensional Crystals from Sequence-Specific Peptoid Polymers. Nat. Mater. 2010, 9, 454-460.
(40) Kirchin, M. A.; Runge, V. M. Contrast Agents for Magnetic Resonance Imaging: Safety Update. Topics in Magnetic Resonance Imaging 2003, 14, 426-435.
(41) Tweedle, M. F.; Hagan, J. J.; Kumar, K.; Mantha, S.; Chang, C. A. Reaction of Gadolinium Chelates with Endogenously Available Ions. Magnetic Resonance Imaging 1991, 9, 409-415.
(42) Mastarone, D. J.; Harrison, V. S.; Eckermann, A. L.; Parigi, G.; Luchinat, C.; Meade, T. J. A Modular System for the Synthesis of Multiplexed Magnetic Resonance Probes. J. Am. Chem. Soc. 2011, 133, 5329-5337.
(43) Minor, D. L.; Kim, P. S. Measurement of the Beta-Sheet-Forming Propensities of Amino Acids. Nature 1994, 367, 660-663.
(44) Bertini, I.; Galas, O.; Luchinat, C.; Messori, L.; Parigi, G. A Theoretical-Analysis of the H-1 Nuclear Magnetic-Relaxation Dispersion Profiles of Diferric Transferrin. J. Phys. Chem.-Us 1995, 99, 14217-14222.
(45) Bertini, I.; Kowalewski, J.; Luchinat, C.; Nilsson, T.; Parigi, G. Nuclear Spin Relaxation in Paramagnetic Complexes of S=1: Electron Spin Relaxation Effects. J. Chem. Phys. 1999, 111, 5795-5807.
(46) Kruk, D.; Nilsson, T.; Kowalewski, J. Nuclear Spin Relaxation in Paramagnetic Systems with Zero-Field Splitting and Arbitrary Electron Spin. Phys. Chem. Chem. Phys. 2001, 3, 4907-4917.
(47) Merbach, A. E. The Chemistry of Contrast Agents in Medical Magnetic Resonance Imaging; Wiley: Chichester, 2001.
(48) Hwang, L.-P.; Freed, J. H. Dynamic Effects of Pair Correlation Functions on Spin Relaxation by Translational Diffusion in Liquids. J. Chem. Phys. 1975, 63, 4017-4025.
(49) Polnaszek, C. F.; Bryant, R. G. Nitroxide Radical Induced Solvent Proton Relaxation: Measurement of Localized Translational Diffusion. J. Chem. Phys. 1984, 81, 4038-4045.
(50) Bertini, I.; Galas, O.; Luchinat, C.; Parigi, G. Computer-Program for the Calculation of Paramagnetic Enhancements Nuclear-Relaxation Rates in Slowly Rotating Systems. J. Magn. Reson. Ser. A 1995, 113, 151-158.
(51) Kowalewski, J.; Kruk, D.; Parigi, G. Nmr Relaxation in Solution of Paramagnetic Complexes: Recent Theoretical Progress for S≥1. Adv. in Inorg. Chem. 2005, 57, 41-104.
(52) Kowalewski, J.; Luchinat, C.; Nilsson, T.; Parigi, G. Nuclear Spin Relaxation in Paramagnetic Systems: Electron Spin Relaxation Effects under near-Redfield Limit Conditions and Beyond. J. Phys. Chem. A 2002, 106, 7376-7382.
(53) Nam, K. T.; Shelby, S. A.; Choi, P. H.; Marciel, A. B.; Chen, R.; Tan, L.; Chu, T. K.; Mesch, R. A.; Lee, B. C.; Connolly, M. D.; Kisielowski, C.; Zuckermann, R. N. Free-floating Ultrathin Two-Dimensional Crystals from Sequence-Specific Peptoid Polymers. Nat. Mater. 2010, 9, 454-460.
(54) Chan, W. C.; White, P. D. Fmoc Solid Phase Peptide Synthesis: a Practical Approach; Oxford University Press: New York, 2000.
(55) Mastarone, D. J.; Harrison, V. S.; Eckermann, A. L.; Parigi, G.; Luchinat, C.; Meade, T. J. A Modular System for the Synthesis of Multiplexed Magnetic Resonance Probes. J. Am. Chem. Soc. 2011, 133, 5329-5337.
(56) Bertini, I.; Galas, O.; Luchinat, C.; Parigi, G. Computer-Program for the Calculation of Paramagnetic Enhancements Nuclear-Relaxation Rates in Slowly Rotating Systems. J. Magn. Reson. Ser. A 1995, 113, 151-158.
(57) Bertini, I.; Kowalewski, J.; Luchinat, C.; Nilsson, T.; Parigi, G. Nuclear Spin Relaxation in Paramagnetic Complexes of S=1: Electron Spin Relaxation Effects. J. Chem. Phys. 1999, 111, 5795-5807.
(58) Kruk, D.; Nilsson, T.; Kowalewski, J. Nuclear Spin Relaxation in Paramagnetic Systems with Zero-Field Splitting and Arbitrary Electron Spin. Phys. Chem. Chem. Phys. 2001, 3, 4907-4917.
(59) Lipari, G.; Szabo, A. Model-Free Approach to the Interpretation of Nuclear Magnetic Resonance Relaxation in Macromolecules: Theory and Range of Validity. J. Am. Chem. Soc. 1982, 104, 4546-4559.
(60) Caravan, P.; Parigi, G.; Chasse, J. M.; Cloutier, N. J.; Ellison, J. J.; Lauffer, R. B.; Luchinat, C.; McDermid, S. A.; Spiller, M.; McMurry, T. J. Albumin Binding, Relaxivity, and Water Exchange Kinetics of the Diastereoisomers of MS-325, a Gadolinium(III)-Based Magnetic Resonance Angiography Contrast Agent. Inorg. Chem. 2007, 46, 6632-6639.

1. A peptide amphiphile comprising:
(a) a hydrophobic non-peptidic segment;
(b) a structural peptide segment;
(c) a charged peptide segment; and
(d) a linker segment.

2. The peptide amphiphile of claim 1, wherein the hydrophobic non-peptidic segment is covalently attached to the structural peptide segment, wherein the structural peptide segment is covalently attached to the charged peptide segment; and wherein the charged peptide segment is covalently attached to the linker segment.

3. The peptide amphiphile of claim 2, wherein the hydrophobic non-peptidic segment is covalently attached to the N-terminus of the structural peptide segment, wherein the C-terminus of the structural peptide segment is covalently attached to the N-terminus of the charged peptide segment; and wherein the C-terminus of the charged peptide segment is covalently attached to the linker segment.

4. The peptide amphiphile of claim 1, wherein hydrophobic non-peptidic segment comprises an acyl chain of 1 to 25 carbons in length.

5. The peptide amphiphile of claim 4, wherein hydrophobic non-peptidic segment comprises a C₁₆ acyl chain.

6. The peptide amphiphile of claim 1, wherein the structural peptide segment is a hydrophobic peptide segment.

7. The peptide amphiphile of claim 6, wherein the hydrophobic peptide segment comprises histidine (H), isoleucine (I), leucine (L), phenylalanine (F), and/or alanine (A) amino acids.

8. The peptide amphiphile of claim 1, wherein the structural peptide segment forms hydrogen bonds and/or other stabilizing interactions with a structural peptide segment from an adjacent peptide amphiphile.

9. The peptide amphiphile of claim 1, wherein the hydrogen bonds and/or other stabilizing interactions result in structure formation that is detectable by circular dichroism and/or microscopy.

10. The peptide amphiphile of claim 9, wherein the structural peptide segment is a α-helix-forming peptide segment.

11. The peptide amphiphile of claim 9, wherein the structural peptide segment is a β-sheet-forming peptide segment.

10. The peptide amphiphile of claim 11, wherein the β-sheet-forming peptide segment comprises 3-8 histidine (H), isoleucine (I), leucine (L), phenylalanine (F), and/or alanine (A) amino acids.

11. The peptide amphiphile of claim 10, wherein the β-sheet-forming peptide segment comprises VVAA (SEQ ID NO: 8).

12. The peptide amphiphile of claim 11, wherein the β-sheet-forming peptide segment comprises VVVAAA (SEQ ID NO: 9).

13. The peptide amphiphile of claim 1, wherein the charged peptide segment comprises acidic and/or basic amino acid residues.

14. The peptide amphiphile of claim 1, wherein the charged peptide segment comprises 1-6 acidic residues selected from glutamate (E) and aspartate (D).

15. The peptide amphiphile of claim 14, wherein the charged peptide segment comprises EE, DD, DE, or DD.

16. The peptide amphiphile of claim 15, wherein the charged peptide segment comprises (X^{a})₃, wherein each X^{a} is an acidic residue.

17. The peptide amphiphile of claim 15, wherein the charged peptide segment comprises EEE.

18. The peptide amphiphile of claim 1, wherein the charged peptide segment comprises 1-6 basic residues selected from histidine (H), arginine (R), and lysine (K).

19. The peptide amphiphile of claim 18, wherein the charged peptide segment comprises (X^{b})₃, wherein each X^{b} is a basic amino acid residue.

20. The peptide amphiphile of claim 1, wherein the structural peptide segment and the charged peptide segment comprise VVVAAAEEE (SEQ ID NO: 1).

20. The peptide amphiphile of claim 20, wherein the structural peptide segment and the charged peptide segment comprise VVVAAAEEEG (SEQ ID NO: 2).

21. The peptide amphiphile of claim 1, wherein the linker segment comprises a moiety capable of forming a covalent bond or stable non-covalent bond with a linking agent.

22. The peptide amphiphile of claim 21, wherein the linker segment comprises a moiety capable of forming a peptide bond with the charged peptide segment.

23. The peptide amphiphile of claim 21, wherein the linker segment is within the charged peptide segment.

24. The peptide amphiphile of claim 21, wherein the linker segment comprises a unnatural amino acid, a reactive natural amino acid, a linker peptoid, an antibody-recognizable epitope, or a ligand.

25. The peptide amphiphile of claim 24, wherein the linker segment comprises a linker peptoid.

26. The peptide amphiphile of claim 25, wherein the linker peptoid is capable of forming a peptide bond with a standard amino acid residue and displays a linkable moiety.

27. The peptide amphiphile of claim 28, wherein the linkable moiety contains one or more functional groups capable of undergoing a huisgen cycloaddition or alkene hydrothiolation.

28. The peptide amphiphile of claim 27, wherein the moiety capable of undergoing a huisgen cycloaddition is an alkyne.

29. The peptide amphiphile of claim 1, comprising: C₁₆-VVVAAAEEEG-(alkyne-modified peptoid) (SEQ ID NO: 3).

30. A composition comprising a peptide amphiphile of one of claims 1-29 and a contrast agent comprising:
(a) (i) a linking moiety covalently attached to (ii) a chelation moiety; and
(b) a metal ion).

31. The composition of claim 30, wherein the linking moiety of the contrast agent is capable of forming a covalent bond or stable non-covalent bond with the linker segment of the peptide amphiphile.

32. The composition of claim 31, wherein the linking moiety of the contrast agent comprises one or more functional groups capable of undergoing a huisgen cycloaddition or alkene hydrothiolation with the linker segment of the peptide amphiphile.

33. The composition of claim 32, wherein the linking moiety of the contrast agent and the linker segment of the peptide amphiphile are: (i) an alkyne and azide, or (ii) an azide and alkyne, respectively.

34. The composition of claim 30, wherein the chelation moiety is selected from the group consisting of EDTA, DTPA, TTHA, DOTA, TAGA, DOTP, DTPA-BMA, DO2P, HP-DO3A, or variants thereof.

35. The composition of claim 34, wherein the chelation moiety comprises HP-DO3A.

36. The composition of claim 30, wherein the metal ion is a paramagnetic metal ion.

37. The composition of claim 36, wherein the paramagnetic metal ion is selected from the group consisting of Mn(II), Gd(III), Dy(III), Ho(III), Er(III), Eu(III), Eu(II), Fe(II), Fe(III), Tb(III), Ce(III), Pr(III), Yb(III), Nd(III), and Tb(IV).

38. The composition of claim 30, wherein the metal ion is a radioactive isotope.

39. The composition of claim 38, wherein the radioactive isotope is selected from the group consisting of In, Ga, or Tc.

40. The composition of claim 30, wherein the metal ion comprises Gd(III).

41. The composition of claim 30, wherein the contrast agent comprises an azide-linked Gd(HP-DO3A) macrocycle.

42. A contrast-agent-labeled peptide amphiphile comprising the reaction product of the contrast agent and peptide amphiphile of claim 30.

43. The contrast-agent-labeled peptide amphiphile of claim 42 comprising C₁₆-VVVAAAEEEG-(peptoid linker)-(Gd(HP-DO3A) macrocycle) (SEQ ID NO: 10).

44. The contrast-agent-labeled peptide amphiphile of claim 43, wherein the peptoid linker and (Gd(HP-DO3A) macrocycle are covalently attached via huisgen cycloaddition or alkene hydrothiolation.

45. The contrast-agent-labeled peptide amphiphile of claim 44, comprising PA1, PA2, PA3, PA4, or variants thereof.

46. A nanofiber comprising the contrast-agent-labeled peptide amphiphiles of one of claims 43-45 and further comprising peptide amphiphiles not-labeled with a contrast agent.

47. The nanofiber of claim 46, wherein the contrast-agent-labeled peptide amphiphiles are Gd(III)-labeled peptide amphiphiles and the peptide amphiphiles not-labeled with a contrast agent are un-Gd(III)-labeled peptide amphiphiles.

48. The composition of claim 46, wherein greater than 50% of the nanofiber is the peptide amphiphiles not-labeled with a contrast agent.

49. A method of monitoring biomaterials in vivo comprising administering a composition including the compound of claim 1 to a human or animal subject as an in vivo implant label and monitoring by a biophysical technique.

50. The method of claim 49, wherein the biophysical technique is magnetic resonance imaging (MRI).

51. The method of claim 49, wherein the biophysical technique is a radioimaging technique.

52. The method of claim 51, wherein the radioimaging technique is Positron emission tomography (PET) or single-photon emission computed tomography (SPECT).

53. The method of claim 49, wherein the biophysical technique is mass spectrometry.
